(19) United States
(12) Patent Application Publication
(10) Pub. No.: US 2026/0266758 A1
(43) Pub. Date: Sep. 10, 2026

(54) GAS SENSOR

(71) Applicant: FARADAIC SENSORS GMBH, Berlin (DE)

(72) Inventors: Alexey YAKUSHENKO, Berlin (DE); Ryan GUTERMAN, Berlin (DE)

(21) Appl. No.: 19/664,861

(22) Filed: Apr. 30, 2026

Related U.S. Application Data
(63) Continuation of application No. PCT/EP2024/080907, filed on Oct. 31, 2024.

(30) Foreign Application Priority Data
Nov. 1, 2023 (GB) 2316747.1

(51) Int. Cl. G01N 27/407; G01N 27/416; G01N 33/00; G03F 7/075; G03F 7/20

(52) U.S. Cl. CPC G01N 27/4073; G01N 27/4163; G01N 33/0027; G03F 7/0757; G03F 7/2002

(57) ABSTRACT

A microfabricated gas sensor for a target gas. The microfabricated gas sensor comprises: an electrolyte, a first electrode and a second electrode. The electrolyte is at least partly provided using photolithography. The first electrode is in contact with a first surface of the electrolyte and is for reducing or oxidising the target gas received through the electrolyte to produce target ions. The second electrode is in contact with a second surface of the electrolyte. With a potential difference applied between the first and second electrodes an amount of electrical current from the first electrode to the second electrode is indicative of at least one of: a concentration of the target gas at the first surface, or a partial pressure of the target gas at the first surface.

### CROSS-REFERENCE TO RELATED APPLICATIONS

This application is a continuation under 35 U.S.C. § 120 of International Application No. PCT/EP2024/080907, filed Oct. 31, 2024 which claims priority to United Kingdom Application No. GB 2316747.1, filed Nov. 1, 2023 under 35 U.S.C. § 119 (a). Each of the above-referenced patent applications is incorporated by reference in its entirety.

### BACKGROUND

A gas sensor produces a response that is related to at least one of: a concentration of a gas, or a partial pressure of a gas that the gas sensor is subject to. Gas sensors may be used to measure environmental conditions, flow rates, pollution, or toxins. It is desirable to: improve the performance of gas sensors, reduce the cost of manufacture of gas sensors, and/or reduce the cost of calibration of gas sensors.

### BRIEF DESCRIPTION OF THE DRAWINGS

FIGS. 1 to 3 show schematically side cross-sections of gas sensors in accordance with examples;

FIG. 4 is a flow diagram of a method of manufacturing a gas sensor in accordance with further examples;

FIG. 5 is a flow diagram of schematic side cross-sections of a gas sensor during the method of FIG. 4;

FIG. 6 is a flow diagram of a method of providing an electrolyte at least partly using photolithography in accordance with further examples;

FIG. 7 is a flow diagram of schematic side cross-sections of a gas sensor during the method of FIG. 6;

FIG. 8 is a flow diagram of a part of a method of manufacturing a gas sensor in accordance with further examples;

FIG. 9 is a flow diagram of schematic side cross-sections of a gas sensor during the method of FIG. 8;

FIG. 10 is a flow diagram of a part of a method of manufacturing a gas sensor in accordance with further examples;

FIG. 11 is a flow diagram of schematic side cross-sections of a gas sensor during the method of FIG. 8;

FIGS. 12 to 14 show schematically top plan views of devices in accordance with further examples;

FIG. 15 is a flow diagram of a method of calibrating a plurality of gas sensors in accordance with further examples;

FIG. 16 shows schematically a top plan view of a plurality of a gas sensor during the method of FIG. 15; and

FIGS. 17 to 25 show schematically structures of polymers in accordance with examples.

### DETAILED DESCRIPTION

A gas sensor of examples herein has a first electrode, a second electrode, and an electrolyte. The electrolyte is at least partly provided using photolithography. An electrical current between the first electrode and the second electrode is at least partly generated by a target ion being formed by an electrochemical reaction between a target gas and the first electrode. The electrical current's magnitude is related to at least one of the concentration of the target gas present at a surface of the first electrode, or a partial pressure of the target gas at the surface of the first electrode. Such gas sensors may be referred to as electrochemical gas sensors.

A gas sensor of examples herein has improved: reliabilities, manufacturing tolerances, improved economies of scale during manufacturing, miniaturization, and/or longevities compared to known gas sensors (e.g., with a liquid electrolyte) because at least partly providing the electrolyte by photolithography allows the electrolyte to be more precisely manufactured than known electrolytes, for example, with smaller variance. Further, at least partly providing the electrolyte by photolithography, in some examples, allows a plurality of gas sensors (e.g., 10,000) to be manufactured in parallel on a single wafer. Thus the cost of manufacturing gas sensors described herein may be lower than for known gas sensors. Further, with the plurality of gas sensors manufactured on a single wafer, the plurality of gas sensors can, in some examples, be calibrated in parallel or in rapid succession with a single method described herein, in contrast to known gas sensors that require more costly and/or slower individual calibration.

Some examples herein significantly reduce manufacturing costs, reduce power requirements, improve production volume, and enable gas sensing in previously impossible scenarios where known gas sensors are too expensive, large, or power hungry. For example, internet-of-things (IoT) devices, wearable devices, and consumer electronics, where billions of small sensors are needed to produce sleek devices economically and sustainably.

In some examples herein the electrolyte, the first electrode, and the second electrode are within a Faraday cage which: prevents or reduces electrical current between the first and second electrodes produced by electromagnetic waves originating from outside the gas sensor, and/or prevents or reduces a noise electrical current between the first electrode and the second electrode. As such, the precision of the sensing of the concentration or partial pressure of the target gas is improved compared to known gas sensors.

In some examples herein, the electrolyte is of a so-called solid-state material, simplifying the structure of the gas sensor compared to gas sensors containing a liquid or gel electrolyte which require components to retain the electrolyte in the gas sensor. Thus in some examples, providing a solid-state electrolyte at least partly by photolithography allows the electrolyte to be smaller than electrolytes of known gas sensors. In some examples, a smaller electrolyte results in the gas sensor not requiring lengthy heating and/or electrochemical preparation cycles associated with larger electrolyte. Thus, gas sensors described herein may have a lower power consumption and/or smaller footprint in a device than known gas sensors.

In contrast to the electrolyte of some examples, electrolytes of known gas sensors may require: a constant temperature, a constant humidity, and complex calibration process during use. This may be because known gas sensors have a liquid electrolyte not formed by photolithography and more susceptible to effects due to changes in temperature, humidity or age. Further, variance in the electrolytes of some examples (for example, variances in size or composition) is reduced compared with known gas sensors as a result of the electrolyte being solid-state. So, the calibration of the gas sensors described herein is simplified, or not necessary compared with known gas sensors. Further, reduced variance in the electrolytes of examples may result in improved reliability and/or longevity of the gas sensor and/or a reduction in the wastage of gas sensors due to imperfections in their manufacture.

The power consumption of the gas sensors herein may be reduced further when using the method of measuring a concentration of a target gas described herein where short time periods of measurement of the concentration of the target gas are used, e.g., instead of conventional continuous measurements.

The gas sensors of some examples herein have a wide range of uses. For example, the reduced size and/or power consumption of gas sensors of some examples herein facilitates the uses known gas sensors would not have been suitable, such as in internet-of-things (IoT) applications where small size and low power consumption are required.

Examples herein relate to at least: gas sensors, methods of manufacturing a gas sensor, devices comprising a gas sensor, methods of calibrating a plurality of gas sensors, or uses of a gas sensor.

In some examples herein at least one of: the gas sensor is a microfabricated, the method of manufacturing a gas sensor is a method of microfabricating a gas sensor, or the plurality of gas sensors are microfabricated. Microfabrication may allow the manufacture of the thousands of gas sensor to be performed in parallel on a single wafer, for example, reducing the cost of manufacture of the gas sensor. Further, microfabrication may allow the gas sensors of examples to be smaller than known gas sensors, e.g., gas sensors manufactured using printing or assembly of separate parts, i.e. not a thin film manufacturing process.

Firstly gas sensors of examples herein are described, descriptions of other examples follow.

### Gas Sensors

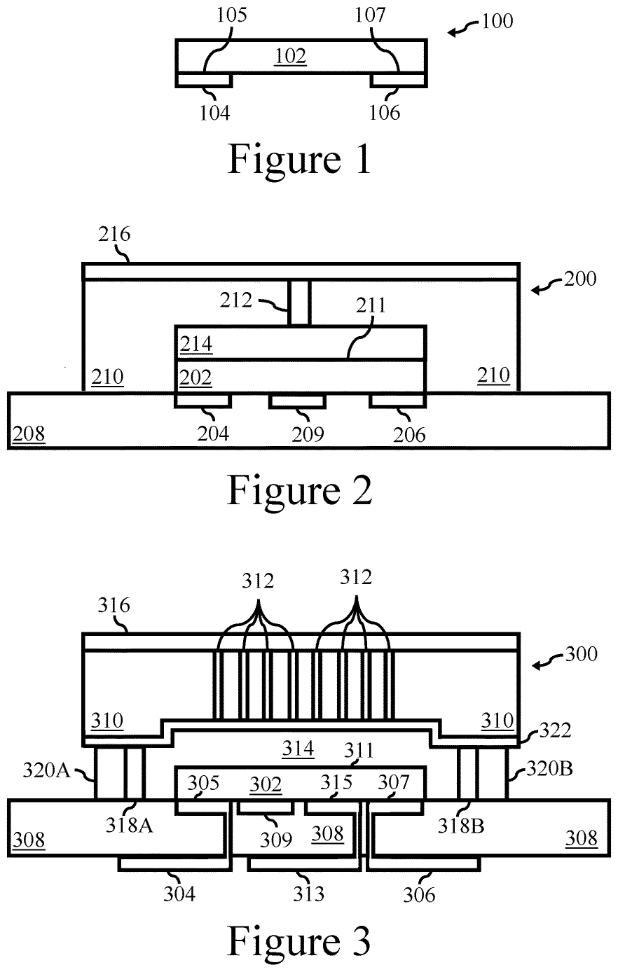

A general introduction to examples herein relating to a gas sensor 100 is now given with reference to FIG. 1. A description of further examples of gas sensors is then given with reference to FIGS. 2 and 3.

The gas sensor 100 comprises an electrolyte 102, a first electrode 104, and a second electrode 106. The electrolyte 102 is at least partly provided using photolithography. Photolithography, for example, involves irradiating a precursor to form the electrolyte from the precursor. In some examples, the electrolyte is provided using at least one of a negative photoresist or positive photoresist. In some examples the electrolyte is deposited and/or patterned using a photolithographic technique, e.g. a lift-off procedure. In some examples the electrolyte is at least partly patterned by photolithography. Further details of photolithography are described later. In some examples, at least one of the gas sensor, the electrolyte, the first electrode or the second electrode is microfabricated.

The first electrode 104 is in contact with a first surface 105 of the electrolyte 102. The second electrode 106 is in contact with a second surface 107 of the electrolyte 102.

The gas sensor 100 is for sensing a target gas, such as the concentration or partial pressure of oxygen. Other target gasses are envisaged such as, carbon dioxide (CO2), carbon monoxide (CO), hydrogen (H2), hydrogen sulfide (H2S), nitrogen oxides (Nox), nitric oxide (NO), nitrogen dioxide (NO2), ammonia (NH3), volatile organic compounds (VOCs), ethylene (C2H4), sulfur dioxide (SO2), water/humidity (H2O), ethanol (C2H5OH), methane (CH4), or combinations thereof. These target gases may also be dissolved gases within a liquid. The first electrode 104 is for reducing or oxidising the target gas received through the electrolyte to produce target ions. The target gas is reduced or oxidised by an electrochemical reaction with the first electrode when a potential difference is applied to the first electrode.

The second electrode 106 is for reducing or oxidising at least one of the electrolyte, a redox additive or an other component of the gas sensor.

In other examples, the second electrode is for reducing or oxidising the target ions produced by the reduction or oxidation of the target gas at the first electrode 104. In some such examples, the target ion is reduced or oxidised by an electrochemical reaction with the second electrode when a potential difference is applied to the first electrode. In some such examples, the target ion is electrostatically attracted to the second electrode and/or electrostatically repelled by the first electrode.

In some examples, the target ion diffuses from the first electrode to the second electrode. In some examples, the electrolyte comprises an ion channel for the target ion to travel along. In other examples, the target ion does not diffuse from the first electrode to the second electrode.

When the target gas is oxygen, it is, e.g., reduced by the first electrode 104 at the first surface 105 to form a target ion. When potential difference is applied between the first electrode 104 and the second electrode 106 an electrical current between the first electrode 104 and the second electrode 106 is indicative of a concentration of the target gas at the first surface 105.

Several configurations of the gas sensor are envisaged; in some examples, the gas sensor is for at least one of the following modes of operation: potentiometric, mixed potential, Amperometric, Coulometric, or electrochemical impedance spectroscopy (EIS). In some such examples electrical circuitry electrically connected to the first electrode and the second electrode is configured for one or more of such modes of operation to determine the concentration of the target gas at the first surface. Such circuitry may also have a power source for applying a potential difference between the first electrode and the second electrode.

The first surface 105 of the electrolyte 102 and the second surface 107 of the electrolyte do not overlap and are spaced from each other. In some examples, the first surface 105 and the second surface 107 are sufficiently separated to reduce or prevent the flow of an electrical current directly from the first electrode 104 and the second electrode 106. The first surface 105 and the second surface 107 are each continuous planar surfaces; however, other forms of the first surface and/or the second surface are envisaged. In other examples at least one of the first surface or the second surface is curved, e.g., to contact a cylindrical first electrode or a cylindrical second electrode. Further, in other examples at least one of the first surface or the second surface comprises a plurality of sub-surfaces which are spaced from each other.

When the gas sensor 100 is in use and there is a potential difference between the first electrode 104 and the second electrode 106, an electrical current from the first electrode 104 to the second electrode 106. The electrical current is, for example measured using an ammeter electrically connected to the first electrode and the second electrode. In other examples, the electrical current is measured using an electrical circuit connected to the first electrode and the second electrode. The electrical current is, for example, no more than 10 Milliamperes (mA). In some examples, the electrical current between the first electrode 104 and the second electrode 106 is from 100 Picoamperes (pA) to 10 Microamperes (μA); in some such examples the electrical current between the first electrode 104 and the second electrode 106 is between 1 Nanoampere (nA) and 1 Microampere (μA). Other electrical current values are envisaged, for example, because the electrical current depends on the size of the first and second electrodes, and the concentration of the target gas at the first surface. In some examples, the electrical current is from the first electrode to the second electrode, or from the second electrode to the first electrode.

In some examples, the electrical current between the first electrode and the second electrode is at least partly generated by: (i) a target ion being formed by the target gas being reduced by the first electrode; and (ii) the second electrode oxidising at least one of: the electrolyte, or a molecule within the electrolyte, such as a redox additive.

In some examples, the electrical current between the first electrode and the second electrode is at least partly generated by: (i) a target ion being formed by the target gas being oxidised by the first electrode; and (ii) the second electrode reducing at least one of: the electrolyte, or a molecule within the electrolyte, such as a redox additive.

In some examples, the electrical current between the first electrode and the second electrode is at least partly generated by: (i) a target ion being formed by an electrochemical reaction between a target gas and the first electrode; (ii) the target ion passing through the electrolyte to the second electrode; and (iii) an electrochemical reaction between the target ion and the second electrode.

The electrical current's magnitude is related to at least one of the concentration of the target gas present at a surface of the first electrode or the partial pressure of the target gas at the first surface. Such gas sensors may be referred to as electrochemical gas sensors.

In some examples, a redox reaction at the second electrode is unknown and/or several redox reactions occur at the second electrode. In some examples, the gas sensor is configured to provide sufficient electrons and/or electron holes at the second electrode for the electrical current's magnitude to be related to at least one of the concentration or partial pressure of the target gas present at a surface of the first electrode, and not be related to the available electrons and/or electron holes at the second electrode. In some examples, a size of the second electrode and/or the electrolyte is chosen so that sufficient electrons and/or electron holes are available.

In some examples, measuring at least one of a partial pressure of the target gas at the first surface 105, or a concentration of the target gas at the first surface 105 comprises at least: (i) exposing the first surface 105 to the concentration of the target gas and/or exposing the first surface 105 to a partial pressure of the target gas; (ii) during (i) applying a potential difference between the first electrode 104 and the second electrode 106; (iii) during (ii) measuring an electrical current value between the first electrode 104 and the second electrode 106; and (iv) calculating the concentration of the target gas at the first surface 105 and/or the partial pressure of the target gas at the first surface 105 from the electrical current value, for example, by using a calibration curve or a lookup table (LUT) of calibration values. In some examples the applying of the potential difference is continuous during the use of the gas sensor. In other examples, the applying of the potential difference in (ii) is for a predetermined period of time, for example, from 1 millisecond (ms) to 10 seconds(s), or from 20 milliseconds (ms) to 200 milliseconds (ms). Further in some such examples, the electrical current value is an integral of an electrical current from the first electrode 104 to the second electrode 106 during the period of time. In some such examples, the above method of measuring the concentration of the target gas at the first surface 105 and/or the partial pressure of the target gas at the first surface 105 is repeated, for example, with a measurement frequency from 0.01 Hertz (Hz) to 100 (Hz). The measurement frequency is how many times the concentration of the target gas is measured by the gas sensor per second. A Hertz (Hz) is equal to a per second (s-1).

In some examples, measuring a concentration of the target gas at the first surface 105 and/or the partial pressure of the target gas at the first surface 105 comprises applying a second potential difference between the first electrode and the second electrode for a second period of time at a different time to (ii) above. In some such examples, the second potential difference is a conditioning pulse for at least one of: pre-conditioning the gas sensor before a measurement or post-conditioning the gas sensor after a measurement. A conditioning pulse may be used to restore a physical or electrochemical property of the gas sensor, for example, a property of the first electrode. Various second potential differences and second time periods are envisaged. In some examples the second potential difference is the reverse polarity of the potential difference of (ii). In other examples the second potential difference is the same as the potential difference of (ii).

In other examples, an electrical value, such as a potential difference value between the first electrode and the second electrode, is related to the concentration of target gas at the surface of the first electrode and/or the partial pressure of the target gas at the first surface. In some such examples, measuring a concentration of the target gas at the first surface and/or the partial pressure of the target gas at the first surface comprises at least: (i) exposing the first surface to the concentration of the target gas and/or exposing the first surface to a partial pressure of the target gas; (ii) during (i) measuring a potential difference value between the first electrode and the second electrode; and (iii) calculating the concentration of the target gas at the first surface and/or the partial pressure of the target gas at the first surface from the potential difference value, for example, by using a calibration curve or a lookup table (LUT) of calibration values. In some such examples the potential difference value between the first electrode and the second electrode is measured using at least one of: a Voltmeter, or an electrical circuit electrically connected to the first electrode and the second electrode.

In some examples the partial pressure of target gas is known (e.g., from a gas pressure sensor), and calculating the concentration of the target gas at the first surface comprises using a calibration curve or a lookup table (LUT) of calibration values for the known partial pressure of the target gas.

In some examples the concentration of the target gas is known, and calculating the partial pressure of the target gas at the first surface comprises using a calibration curve or a lookup table (LUT) of calibration values for the known concentration of the target gas.

In some examples, the partial pressure of the target gas at the first surface is used to determine a flow rate of the target gas over the gas sensor.

In some examples, measuring a concentration of the target gas at the first surface and/or partial pressure of the target gas at the first surface comprises electrochemical impedance spectroscopy (EIS). In some such examples, measuring the concentration of the target gas at the first surface and/or partial pressure of the target gas at the first surface comprises: (i) applying an alternating potential difference between the first electrode and the second electrode; and (ii) measuring an impedance between the first electrode and the second electrode. In some such examples, (i) comprises applying a sinusoidal potential difference between the first electrode and the second electrode. The electrical current between the first electrode and the second electrode may have a different amplitude and/or phase than the sinusoidal potential difference between the first electrode and the second electrode. Other waveforms of the potential difference are envisaged such as square, saw-tooth, or pulses. By varying the frequency of the alternating potential difference, the impedance between the first electrode and the second electrode can be measured at each frequency of the alternating potential difference to determine an impedance spectrum (a spectrum of frequency of the alternating potential difference against impedance). In some examples, the impedance spectrum is used to at least one of: determine a property of the gas sensor, partial pressure of the target gas at the first surface, or the concentration of the target gas at the first surface.

In some examples, the power consumption of the gas sensor when measuring the concentration of the target gas and/or partial pressure of the target gas at the first surface is from 1 Milliwatt (mW) to 1 Microwatt (μW). Other values are envisaged and it is envisaged that the power consumption of the gas sensor will depend on several factors, such as the measurement frequency. For example: with a measurement frequency of 100 Hertz (Hz) the power consumption of the gas sensor is 1 Milliwatts (mW); with a measurement frequency of 1 Hertz (Hz) the power consumption of the gas sensor is 20 Microwatts (μW); or with a measurement frequency of 0.017 Hertz (Hz) the power consumption of the gas sensor is 1 Microwatt (μW). A Watt (W) is equal to a kilogram metre squared per second cubed (kg·m2·s-3).

The electrolyte 102 is inorganic; in other examples the electrolyte is at least one of organic, inorganic, polymeric, or ceramic. The electrolyte 102 is solid-state and may be considered as a solid-state electrolyte. In other examples the electrolyte is a combination of solid-state with at least one of a liquid or a gel.

In some examples the electrolyte is a phase-separated material and/or contains ion channels.

In some examples the electrolyte comprises a polymer comprising: a silicone, an anion and a cation. At least one of the cation or the anion is covalently bonded to the silicone. In other examples, the polymer does not comprise a silicone. Example of the polymer are described in more detail later.

A description of a gas sensor of further examples is now given in relation to FIG. 2. Where a feature in relation to FIG. 2 corresponds with a feature described using FIG. 1, a reference numeral is used which is 100 greater than the corresponding reference numeral used for FIG. 1 (e.g., 102 in FIG. 1 is 202 in FIG. 2); corresponding descriptions for such features apply here also. The gas sensor 200 of FIG. 2 is the same as the gas sensor 100 of FIG. 1, except for the following differences.

The gas sensor 200 comprises a substrate 208. The substrate 208 supports the electrolyte 202, the first electrode 204, and the second electrode 206. The first electrode 204 and the second electrode 206 are at least partly embedded within the substrate and in contact with the substrate. Other configurations of the first electrode and/or the second electrode are envisaged. The gas sensor 200 comprises a cap and a cavity 214. The cavity 214 is between the cap and a sensing surface 211 of the electrolyte 202. The cap comprises a cap substrate 210 and a gas channel 212. The cap substrate 210 is directly attached to the substrate 208. In other examples the cap substrate is indirectly attached to the substrate. The gas channel 212 is through the cap substrate 210 and is from the cavity 214. The gas sensor 200, comprises a gas-permeable layer 216. The gas permeable layer 216 is on the cap substrate 210. The gas channel 212 is from the gas-permeable layer 216 to the cavity 214. In other examples, the gas channel is at least partly through the gas-permeable layer.

The gas sensor 200 comprises a heater 209. The substrate supports the heater 209. The heater 209 is at least partly embedded within the substrate 208. Other arrangements of the heater and substrate are envisaged. In some examples, the method of measuring the concentration of the target gas at the first surface 205 using the gas sensor comprises the heater heating at least one of the gas sensor 200 or the electrolyte 202. The heater 209 is in contact with the electrolyte 202. In other examples the heater is not in contact with the electrolyte, for example, there is at least one of: a heat spreader, heat pipe or thermal conductor between the heater and the electrolyte. In some such other examples the heater provides even heating of the electrolyte and/or faster heating of the electrolyte. In other examples, the gas sensor does not comprise a heater.

In other examples not illustrated, the gas sensor comprises a cooler, and the method of measuring the concentration of the target gas at the first surface using the gas sensor comprises the cooler cooling at least one of the gas sensor or the electrolyte. In some such examples the cooler is in contact with the electrolyte. In other examples the cooler is not in contact with the electrolyte, for example, there is at least one of: heat spreader, heat pipe and/or thermal conductor between the cooler and the electrolyte. In some such other examples, the cooler provides even cooling of the electrolyte and/or faster cooling of the electrolyte. In other examples, the gas sensor does not comprise a cooler.

In some examples a temperature of the electrolyte during use can be lower than known gas sensors. This may be because known gas sensors have a liquid electrolyte not formed by photolithography which may freeze at low temperatures, resulting in known gas sensors becoming inoperable due to, for example, the electrolyte becoming impermeable to the target gas, and/or an increase in the ionic resistance of the electrolyte. The temperature of the electrolyte or the gas sensor during a use is, in some examples herein is at least one of, from 194 Kelvin (K) to 573 Kelvin (K), from 220 Kelvin (K) to 573 Kelvin (K), or from 220 Kelvin (K) to 453 Kelvin (K). In some examples, the temperature is no less than 194 Kelvin (K), in some such examples, the temperature is no less than 220 Kelvin (K). Some gas sensors herein operate in a wider range of temperatures than other known gas sensors allowing the use of gas sensors herein in previously unachievable applications and/or environments. In some examples the gas sensor comprises a temperature probe for measuring at least one of: the temperature of the electrolyte or the temperature of the gas sensor.

In some examples a humidity at the electrolyte during use can be at least one of lower or higher than known gas sensors. Further, the electrolyte and/or gas sensor of examples herein may be at least partly submerged or submerged in a liquid during use, in contrast to known gas sensors. In some examples, the gas sensor herein is configured for use in a humidity from 0 percent to 100 percent. This may be because known gas sensors have a liquid electrolyte not formed by photolithography. The electrolyte of known gas sensors may become inoperable in such conditions, for example, because of the electrolyte becoming impermeable to the target gas, the electrolyte being dissolved in the liquid, the electrolyte being mixed with the liquid, the electrolyte evaporating/drying out, and/or an increase in the ionic resistance of the electrolyte. Some gas sensors herein operate in a wider range of humidities than other known gas sensors allowing the use of gas sensors herein in previously unachievable applications and/or environments. In some examples the gas sensor comprises a humidity probe for measuring at least one of: the humidity at the electrolyte or the humidity at the gas sensor.

A description of a gas sensor of further examples is now given in relation to FIG. 3. Where a feature in relation to FIG. 3 corresponds with a feature described using FIG. 2, a reference numeral is used which is 100 greater than the corresponding reference numeral used for FIG. 2 (e.g., 202 in FIG. 2 is 302 in FIG. 3); corresponding descriptions for such features apply here also. The gas sensor 300 of FIG. 3 is the same as the gas sensor 200 of FIG. 2, except for the following differences.

The gas sensor 300 comprises a plurality of gas channels 312. Each of the plurality of the gas channels 312 is from the cavity 314 to the gas permeable layer 316. The first electrode 303 and the second electrode 306 are through the substrate 308. The gas sensor comprises a third electrode 313. The third electrode 313 is in contact with a third surface 315 of the electrolyte 302. The third surface 315 does not overlap with the first surface 305 or the second surface 307. The third surface 315 is spaced from the first surface 305 and the second surface 307. In some examples, the third surface 315 is sufficiently spaced from the first surface 305 and the second surface 307 to reduce or prevent the flow of an electrical current from the third electrode 313 to the first electrode 304 or the second electrode 306. Similarly to the first surface 305 and the second surface 307, the third surface 315 is a continuous planar surface; however, other forms are envisaged. In other examples the third surface is curved, e.g., to contact a cylindrical electrode. Further, in other examples at least third surface comprises a plurality of sub-surfaces which are spaced from each other.

The first electrode 304 is a first working electrode 304 and the third electrode 313 is a second working electrode 313. The first working electrode 304 is for sensing a first target gas and the second working electrode is for sensing a second target gas different to the first target gas. In other examples, the gas sensor comprises at least one of: a reference electrode in contact with the electrolyte; a diagnostic electrode in contact with the electrolyte; or a grounding electrode in contact with the electrolyte. Other electrodes are envisaged.

The cap comprises an electrically conductive layer 322 between the cap substrate 310 and the cavity 314. The electrically conductive layer 322 and the substrate 308 are electrically connected. The electrically conductive layer 322 is electrically connected to two electrically conductive portions 318A and 318B. The electrically conductive portions 318A and 318B are both electrically connected the substrate 308. In some examples the electrically conductive portions 318A and 318B are electrically earthed by the substrate 308. In other example the electrically conductive layer is directly attached to the substrate and directly electrically connected to the substrate. The combination of the electrically conductive layer 322, the electrically conductive portions 318A and 318B and the substrate 308 provide a Faraday cage, the electrolyte 302 within the Faraday cage. In other examples, the electrolyte is within a Faraday cage provided by other components or combinations or components. For example, in other examples, the cap substrate is electrically conductive and electrically bonded to the substrate. So, the cap substrate and the substrate provide a Faraday cage around the electrolyte. In other examples, the Faraday cage is a partial Faraday cage. In other examples, the cap substrate is electrically connected to the substrate by one electrically conductive portion. The cap is supported by two stand offs 320 A and 320B. In some examples, such as those of FIG. 3, the stand offs 320A and 320B prevent the cap substrate 310 colliding with the substrate 308.

### Methods of Manufacturing a Gas Sensor

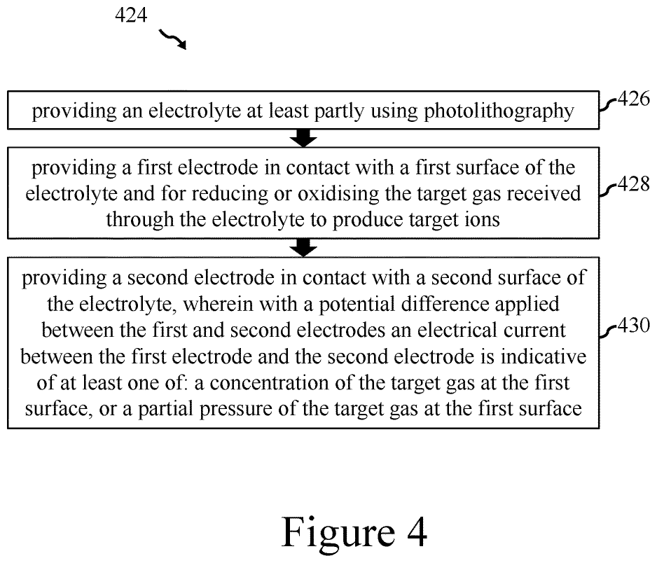

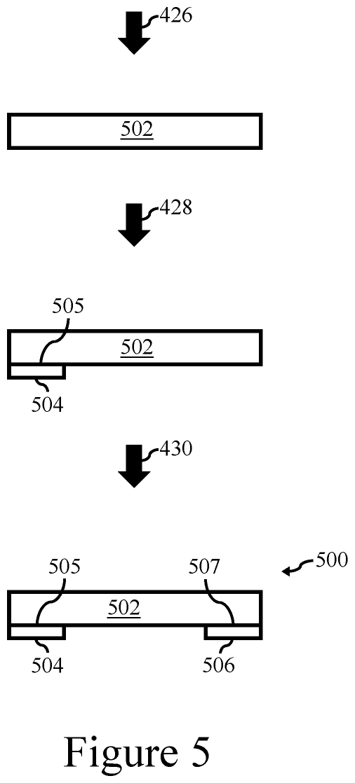

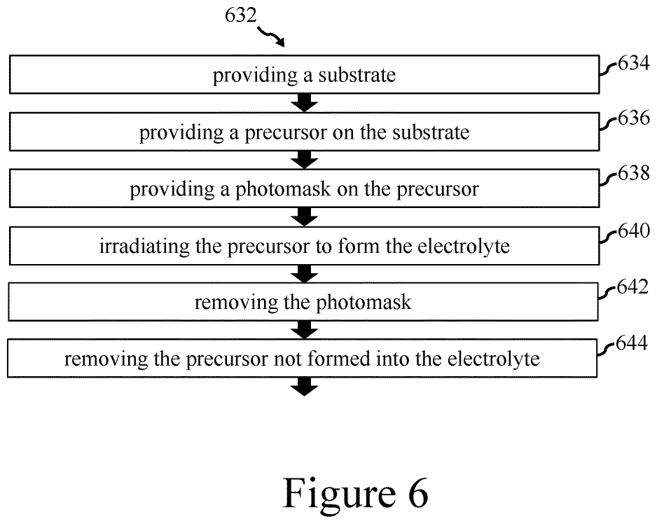

A description of examples herein relating a method of manufacturing a gas sensor is now given in relation to FIGS. 4 and 5. A description of methods of manufacturing a gas sensor of further examples is then given with reference to FIGS. 6 to 11.

FIG. 4 is a flow diagram of an example method of manufacturing a gas sensor. FIG. 5 schematically shows a flow diagrams of respective side cross-sections of the method of FIG. 4.

Where a feature in relation to FIG. 5 corresponds with a feature described using FIG. 3, a reference numeral is used which is 200 greater than the corresponding reference numeral used for FIG. 3 (e.g., 302 in FIG. 3 is 502 in FIG. 5); corresponding descriptions for such features apply here also. Where a feature in relation to FIG. 5 corresponds with a feature described using any of FIG. 4, the same reference numeral is used.

In the examples of FIGS. 4 and 5, the method 424 of manufacturing a gas sensor 500 comprises providing 426 an electrolyte 502 at least partly using photolithography. In some examples the electrolyte is entirely provided by photolithography; in other examples, providing the electrolyte comprises other process or techniques, for example, etching or curing. Other techniques and processes are envisaged and detailed later. Then the method 424 comprises providing 428 a first electrode 504. The first electrode 502 is in contact with a first surface 505 of the electrolyte 502 and is for reducing or oxidising the target gas received through the electrolyte 502 to produce target ions. Next, the method 424 comprises providing 430 a second electrode. The second electrode 506 is in contact with a second surface 507 of the electrolyte 502. With a potential difference applied between the first electrode 504 and the second electrode 506 an electrical current flows from the first electrode 504 to the second electrode 506 and is indicative of a concentration of the target gas at the first surface 505. In other examples, the steps of the method of manufacturing the gas sensor are conducted in a different order. For example, in other methods of examples: the second electrode is provided, then the first electrode is provided, then the electrolyte is provided at least partly using photolithography.

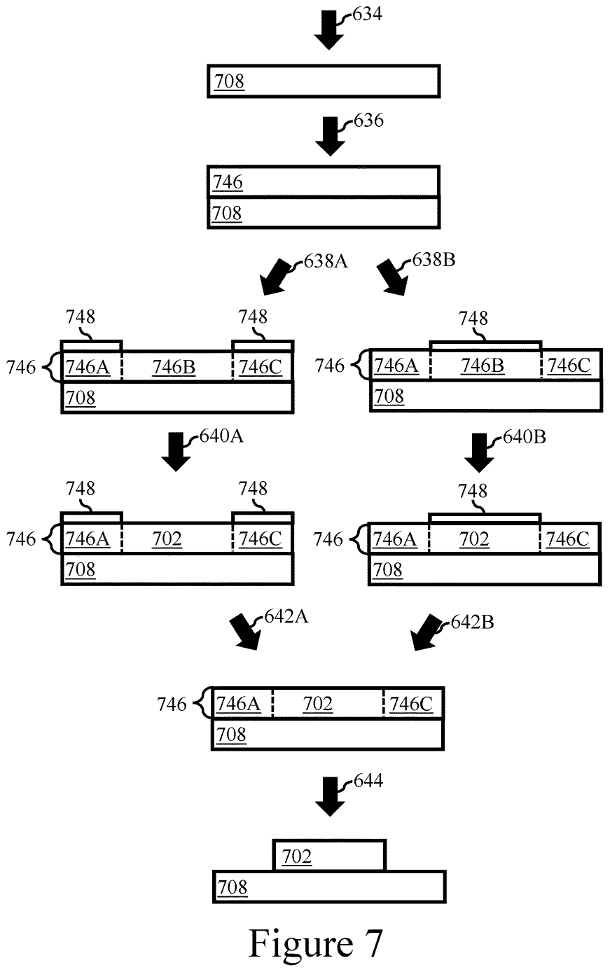

A description of a method 632 of providing an electrolyte at least partly using photolithography of further examples is now given in relation to FIGS. 6 and 7. The method 632 is, for example, at least part of the providing 426 an electrolyte at least partly using photolithography described in relation to FIGS. 4 and 5. For example, the method 632 of such examples may be followed by at least: providing a first electrode in contact with a first surface of the electrolyte and for reducing or oxidising the target gas received through the electrolyte to produce target ions; and providing a second electrode in contact with a second surface of the electrolyte and for reducing or oxidising the target ions, where with a potential difference applied between the first and second electrodes an electrical current between the first electrode and the second electrode is indicative of a concentration of the target gas at the first surface as described in relation to FIGS. 4 and 5.

FIG. 6 is a flow diagram of providing an electrolyte at least partly using photolithography. FIG. 5 shows schematically a flow diagram of respective side cross-sections of the gas sensor during the method of FIG. 6.

Where a feature in relation to FIG. 7 corresponds with a feature described using FIG. 3, a reference numeral is used which is 400 greater than the corresponding reference numeral used for FIG. 3 (e.g., 302 in FIG. 3 is 702 in FIG. 7); corresponding descriptions for such features apply here also. Where a feature in relation to FIG. 7 corresponds with a feature described using FIG. 6, the same reference numeral is used.

In the examples of FIGS. 6 and 7, the method 632 comprises providing 634 a substrate 708. Then the method 632 comprises providing 636 a precursor 746 on the substrate 708. The precursor 746 is in contact with the substrate 708. In other examples the precursor is not in contact with the substrate, for example, there is a layer or portion between the precursor and the substrate. In some examples, providing the precursor on the substrate comprises printing the precursor on to the substrate, for example using an inkjet printer; however other printing methods are envisaged. In some examples, the printed precursor or electrolyte is resistant to deformation, deswelling, or creep due to changes in temperature or the passage of time. Printing also allows for the production of a precursor or an electrolyte in a manner which is compatible with other common microfabrication techniques, which in turn, in some examples simplifies the inclusion of the electrolyte in a microchip or microelectronics. In some examples the printed electrolyte is resistant to solvents and chemical vapours. Resistance to solvents and chemical vapours, for example, allows the electrolyte to be exposed to other common microfabrication techniques without reducing the ion conductivity of the ion conductor.

Other techniques envisaged for providing the resist mixture on a substrate include for example drop casting, dip coating, spray coating, roll coating, painting, doctor blading, condensation, and imprint lithography.

The method 632 then comprises providing 638, 638A, 638B a photomask 748 on the precursor 746, and then irradiating 640 the precursor 746 to form the electrolyte 702. In some examples, the precursor is heated during the irradiation of the precursor 746.

In some examples of the method 623, the precursor 746 is a negative photoresist. In such examples, providing 638A a photomask 748 on the precursor 746 comprises providing the photomask 748 on a first potion 746A of the precursor 746 and on a third portion 746C of the precursor. The photomask 748 is not on a second portion 746B of the precursor 746. The second portion 746B of the precursor 746 is between the first portion 746A of the precursor 746 and the third portion 746C of the precursor 746. The method 623 where the precursor 746 is a negative photoresist then comprises irradiating 640A the second portion 746B of the precursor 746 to form the electrolyte 702 from the second portion 746B of the precursor 746. The photomask prevents or reduces irradiation of the first portion 746A of the precursor 746 and the third portion 746C of the precursor 746.

In some examples of the method 623, the precursor 746 is a positive photoresist. In such examples, providing 638B a photomask 748 on the precursor 746 comprises providing the photomask 748 on a second potion 746B of the precursor 746. The photomask 748 is not on a first portion 746A of the precursor 746 or a third portion 746C of the precursor 746. The second portion 746B of the precursor 746 is between the first portion 746A of the precursor 746 and the third portion 746C of the precursor 746. The method 623 where the precursor 746 is a positive photoresist then comprises irradiating 640B the first portion 746A of the precursor 746 and the third portion 746C of the precursor 746 to form the electrolyte 702 from the second portion 746B of the precursor 746. The photomask prevents or reduces irradiation of the second portion 746B of the precursor 746.

Other forms of photoresists, such as an image reversal photoresist are envisaged.

In some examples irradiating the precursor comprises exposing the precursor to at least one of an electron beam or electromagnetic radiation.

In some examples, photolithography does not comprise providing a photomask on the precursor or removing the photomask. For example, the precursor is selectively irradiated using projector, and/or by modulating the source of radiation, such as by rastering a laser across the precursor whilst selectively controlling the power of the laser. In some examples the precursor is heated or annealed to cause crosslinking. In some examples this is achieved with thermal initiators, in other examples this is achieved without thermal initiators. In some examples where thermal initiators are not used, crosslinking is achieved at temperatures of 423 Kelvin and above. In some examples where thermal initiators are used, crosslinking is achieved at room temperature (298 Kelvin), in further examples where thermal initiators are used, this is achieved at a temperature between 323 Kelvin and 393 Kelvin. Some examples undergo heating at any point in manufacture which allows the facilitation of chemical reactions and/or the removal of volatiles from the electrolyte.

Then the method 623 for both a positive photoresist and a negative photoresist then comprises removing 642, 642A, 642B the photomask 748. Next, the method 623 comprises removing the precursor 746A and 746B not formed into the electrolyte 702.

In some examples, the precursor not formed into the electrolyte is removed by etching. Etching, for example, is achieved chemically and/or radiatively. Etching, for example, is at least one of dry etching or wet etching. Etching, for example, gives the patterning of the electrolyte on the substrate.

In some examples, such as some examples when the precursor is printed onto the substrate, it is not always necessary to remove the precursor that has not been formed into the electrolyte, for example, as the printing allows the patterning of the electrolyte on the substrate. In some examples the electrolyte is patterned on the substrate. This allows the incorporation of components (such as potentially highly leachable components) which would otherwise degrade or be removed if they were exposed to a developer for removal of precursor that has not been formed into the electrolyte.

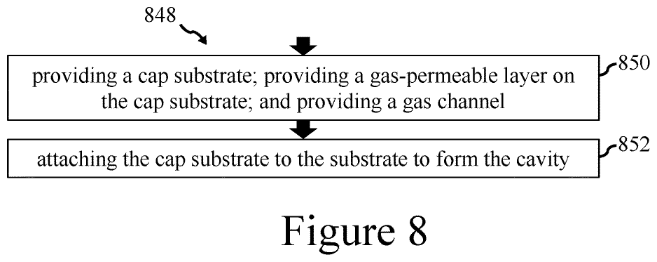

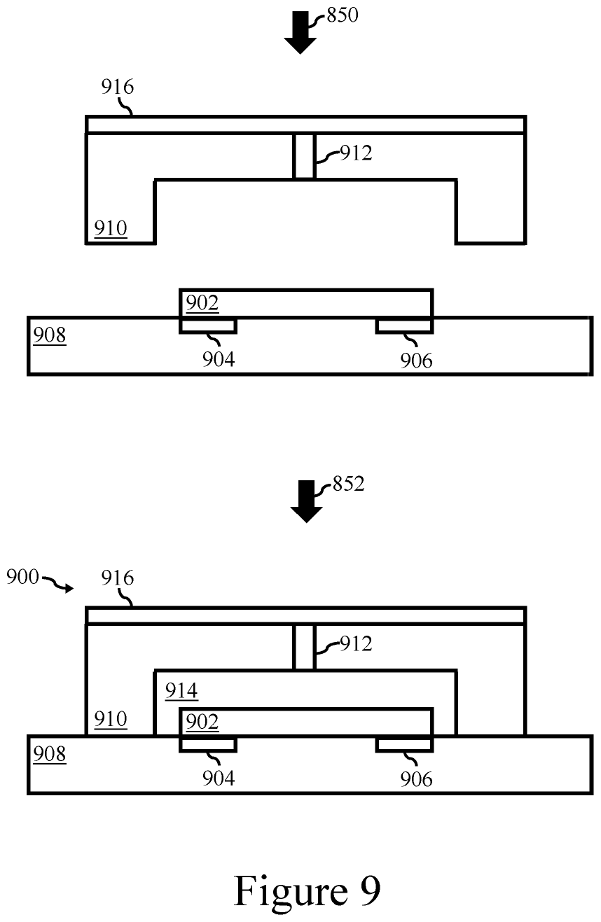

A description of a part of a method of manufacturing a gas sensor of further examples is now given in relation to FIGS. 8 and 9. The part 848 of a method of manufacturing a gas sensor 900 is, for example, part of the method of manufacturing as gas sensor described in relation to FIGS. 4 and 5. For example, the part 848 of the method of manufacturing a gas sensor 900 of such examples may be preceded by the method as described in relation to FIGS. 4 and 5.

FIG. 8 is a flow diagram of the part 848 of a method of manufacturing a gas sensor. FIG. 9 shows schematically a flow diagram of respective side cross-sections of the gas sensor during the part 848 of FIG. 8.

Where a feature in relation to FIG. 9 corresponds with a feature described using FIG. 3, a reference numeral is used which is 600 greater than the corresponding reference numeral used for FIG. 3 (e.g., 302 in FIG. 3 is 902 in FIG. 9); corresponding descriptions for such features apply here also. Where a feature in relation to FIG. 9 corresponds with a feature described using FIG. 8, the same reference numeral is used.

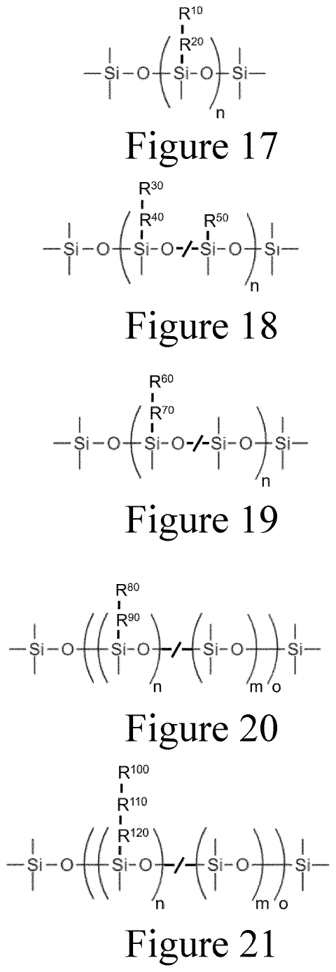

In the examples of FIGS. 8 and 9, the part 848 of a method of providing a gas sensor comprises providing 850 a cap substrate 910; providing 850 a gas-permeable layer 916 on the cap substrate 910; and providing 850 a gas channel. The part 848 then comprises attaching 852 the cap substrate 910 to the substrate 908 to form the cavity 914. The gas channel 912 is from the gas permeable layer 916 to the cavity 914. In some examples, such as those of FIGS. 17 to 19, attaching 852 the cap substrate 910 to the substrate 908 is the final technique or process in the method of manufacturing the gas sensor 900. In other examples, attaching the cap substrate to the substrate is followed by other techniques or processes. In some examples, attaching the cap substrate to the substrate comprises at least on one of: a flip-chip process, a pick-and-place process, or tape-automated bonding (TAB),

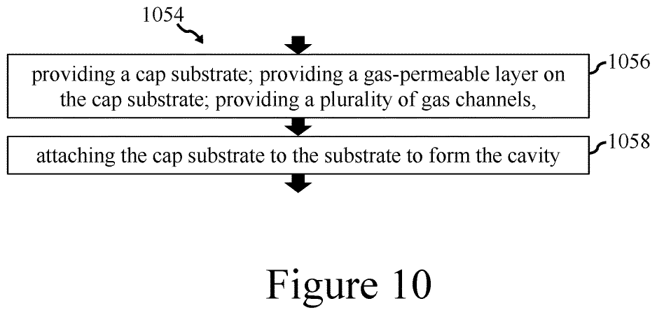

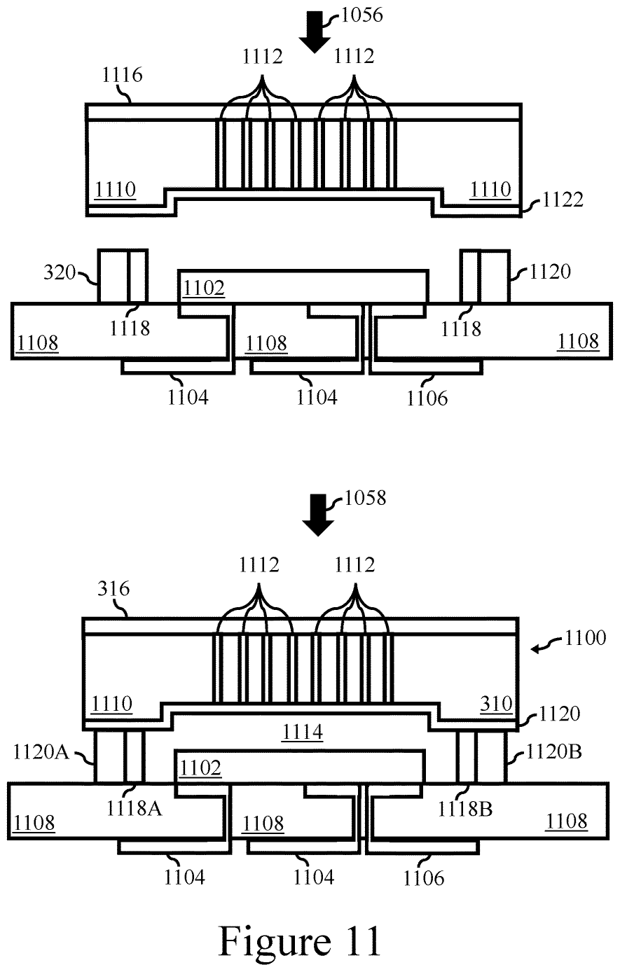

A description of a part 1054 of a method of manufacturing a gas sensor of further examples is now given in relation to FIGS. 10 and 11. The part 1054 of a method of manufacturing a gas sensor 1100 is, for example, part of the method of manufacturing as gas sensor described in relation to FIGS. 4 and 5. For example, the method 1054 of manufacturing a gas sensor 1100 of such examples would be preceded by the method as described in relation to FIGS. 4 and 5.

FIG. 10 is a flow diagram of the part 1054 of a method of manufacturing a gas sensor. FIG. 11 shows schematically a flow diagram of respective side cross-sections of the gas sensor during the part 1054 of FIG. 10.

Where a feature in relation to FIG. 11 corresponds with a feature described using FIG. 3, a reference numeral is used which is 800 greater than the corresponding reference numeral used for FIG. 3 (e.g., 302 in FIG. 3 is 1102 in FIG. 11); corresponding descriptions for such features apply here also. Where a feature in relation to FIG. 11 corresponds with a feature described using FIG. 10, the same reference numeral is used.

In the examples of FIGS. 20 to 22, the part 1054 comprises providing 1056 a cap substrate 1110; providing 1056 a gas-permeable layer 1116 on the cap substrate 1110; and providing a plurality of gas channels 1112. The part 1054 also comprises providing an electrically conductive layer 1122. The part 1054 then comprises attaching 1058 the cap substrate 1110 to the substrate 1108 to form the cavity 1114.

The electrically conductive layer 1122 is between the cap substrate 1110 and the cavity 1114. The electrically conductive layer 1122 and the substrate 1108 are electrically connected. The electrically conductive layer 322 is electrically connected to the substrate 1108 by two electrically conductive portions 1118A and 1118B. The electrically conductive portions 1118A and 1118B are electrically earthed by the substrate 1108. The electrically conductive layer 1120 is electrically earthed via the electrically conductive portions 1118A and 1118B. The combination of the electrically conductive layer 322, the electrically conductive portions 318A and 318B and the substrate 308 provide a Faraday cage, the electrolyte 1102 within the Faraday cage. The cap substrate 1110 is attached to the substrate 1108 by two stand offs 1120 A and 1120B. In some examples, such as those of FIG. 3, the stand offs 1120A and 1120B also prevent the cap substrate 310 colliding with the substrate 1108 during the method of manufacturing the gas sensor. In some examples the stand offs 1120A and 1120B allow control of the separation between the substrate and the cap substrate and/or allow control of the size of the cavity.

### Devices

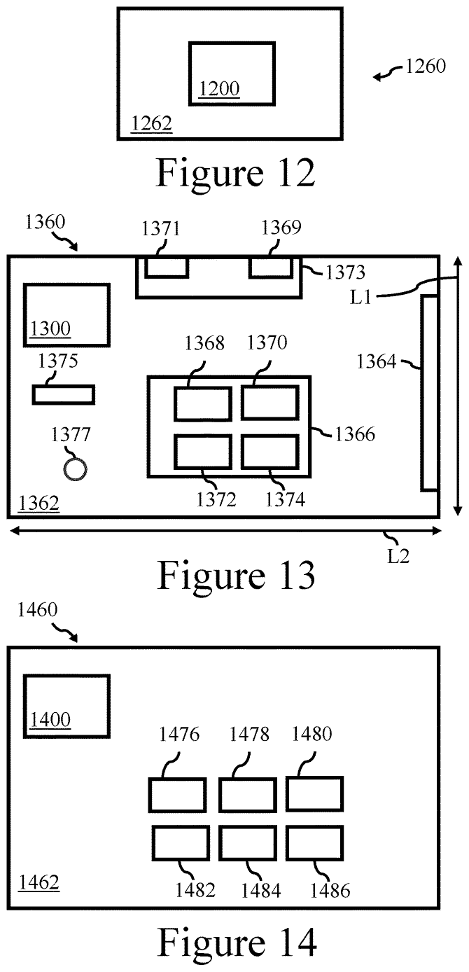

A description of examples herein relating to devices is now given in relation to FIG. 12. A description of further examples relating to devices is then given in relation to FIGS. 13 and 14. The devices of examples comprise a gas sensor as described herein.

FIG. 12 is a top plan view of the device 1260. Where a feature in relation to FIG. 12 corresponds with a feature described using any of FIG. 3, a reference numeral is used which is 900 greater than the corresponding reference numeral used for FIG. 3 (e.g., 300 in FIG. 3 is 1200 in FIG. 12); corresponding descriptions for such features apply here also.

The device 1260 comprises a board 1262, and a gas sensor 1200 of examples herein supported by the board 1262. The gas sensor 1200 is electrically connected to the board 1262. The board 1262 is a printed circuit board (PCB), other boards are envisaged. In some examples, the board comprises at least one of: fiberglass, polyimide, poly(1,1,2,2-tetrafluoroethylene) (PTFE), a flexible polymer, a ceramic, a glass, silicon (Si), paper, fabric, or a biodegradable polymer. In some examples further components of the device are at least one of: part of the board or integrated within the board. In some examples, portions of the board are at least one of: an antenna, or an electrode. In some examples at least one of the first electrode or the second electrode of the gas sensor is integrated with the board. In some examples passive components may be designed as integrated with, embedded with or buried within the board. A passive component herein is a component that does not require electrical power when in use. In some examples the integration of components with the board allows the size of the device to be reduce and/or enhances the stability of the device. In some examples, the board is at least one of flexible and/or flexibly rigid. In some such examples the flexibility of the board allows to the size of the device to be reduced, for example by folding the board.

In some examples, the device comprises the gas sensor herein and packaging, e.g. for a particular application. Packaging can therefore be considered, e.g., as any element, structure or component assembled with the gas sensor to provide a commercial product. Such a packaged gas sensor may therefore simply be an appropriately housed gas sensor for connection to appropriate circuitry for then assembling into a more complex device which utilises the gas sensor. Or in other examples, the packaged gas sensor may refer to such a complex device, such as a standalone device usable by an end user. In some such examples, the gas sensor is packaged. In some examples, the packaging is a traditional package and/or wafer-scale package. For example, the gas sensor could be packaged as described herein in relation to any of FIGS. 12 to 14. However, other packaging is envisaged. The packaging may depend on the complexity of the device, for example, a device for integration with other electronics may have different packaging to a device for standalone operation.

A description of a device of further examples is now given in relation to FIG. 13. The device 1360 comprises a gas sensor 1300 as described herein.

FIG. 13 is a top plan view of the device 1360. Where a feature in relation to FIG. 24 corresponds with a feature described using FIG. 23, a reference numeral is used which is 100 greater than the corresponding reference numeral used for FIG. 12 (e.g., 1200 in FIG. 12 is 1300 in FIG. 13); corresponding descriptions for such features apply here also.

The device 1360 comprises a board 1362, and a gas sensor 1300 of examples herein supported by the board 1362. The board 1462 comprises four layers of polyimide and is suitable for components to be mounted to either side of the board. The width of the device (L1) is 6 millimetres (mm), the length of the device (L2) is 12 millimetres (mm) the depth of the device is 2 millimetres (mm). The width, length, and depth of the device are all mutually perpendicular. The device 1360 comprises a flexible printed circuit connector (FPCC) 1364. In some examples, the FPCC comprises 10 pads for electrical connections. The FPCC 1364 is at an edge of the device 1360. The device 1360 comprises a wafer layer chip scale package (WL-CSP) 1366. The WL-CSP 1366 comprises a multiplexer 1368, an operational amplifier 1370, a microcontroller 1372, and a power regulator. The device comprises an assembly of passive components 1375. The assembly of passive components, for example, comprises resistors and/or capacitors. The device 1360 comprises a sensor module 1373 comprising sensing element. The sensor module 1373 of FIG. 24 comprises a humidity sensor element 1371 and a temperature sensor element 1369. The sensing elements 1369 and 1371 are attached to the board 1362 by an isotropic conductive adhesive. In other examples, the sensor elements are integrated with the board. In some examples the sensor module is covered by a shield, for example a metal layer.

The device 1360 comprises an alignment feature 1377. The alignment feature 1377 is a hole and is, for example, for aligning the device with another product. In other examples the alignment feature is at least one of: a contact pad, a mechanical feature for fixation of the device, a slot, an edge, a ridge, or a post. In some examples the device is integratable with another product, for example by socket installation, soldering, or adhesive bonding, alternative methods of integration are envisaged. In some examples, the device is integrated with another product using 3D moulded interconnected devices (3D-MID) technology.

A description of a device of further examples is now given in relation to FIG. 25. The device 1460 comprises a gas sensor 1400 as described herein.

FIG. 14 is a top plan view of the device 1460. Where a feature in relation to FIG. 14 corresponds with a feature described using any of FIG. 13, a reference numeral is used which is 100 greater than the corresponding reference numeral used for FIG. 13 (e.g., 1300 in FIG. 13 is 1400 in FIG. 14); corresponding descriptions for such features apply here also.

The device 1460 comprises a board 1462, and a gas sensor 1400 of examples herein supported by the board 1462. The device comprises an application-specific integrated circuit (ASIC) 1476, and a power management integrated circuit (PMIC) 1478. In some examples, the PMIC stabilises the required potential difference for the device. The device 1460 comprises a communications module 1480. The communications module, for example, comprises at least one of an antenna, an antenna for short-range wireless communication, or a radio frequency identification (RFID) system. The communications module 1480 is for example, for at least one of: in-device diagnostics, identification, or calibration of the sensor. The device 1460 comprises a memory module 1482. The memory module, for example comprises at least one of: circuitry to manage the sensor's operation, memory for storing calibration parameters, memory for storing usage history, memory for storing sensor identification, internal memory, primary memory random access memory (RAM), dynamic random access memory (DRAM), static random access memory (SRAM), read-only memory (ROM), programmable read-only memory (PROM), or a cache. The device 1460 comprises an additional sensor 1484. The additional sensor 1484 is, for example, at least one of: a temperature sensor, a thermistor, a semiconductor sensor, an integrated sensor, a humidity sensor, a pressure sensor, a gas flow rate sensor, a dew point sensor, an accelerometer, a sensors for gas that is not the target gas, a sensor for a chemical substance, a microchip comprising a plurality of discrete sensing elements. The device 1460 comprises an analogue-to-digital converter 1486 for converting an analogue signal from the gas sensor 1400 into a digital signal.

In some examples, the device comprises the gas sensor described herein and an ASIC. In some such examples the gas sensor and the ASIC are both on a board. In other such examples, the gas sensor and the ASIC are integrated together.

In other examples of the device, the device comprises at least one of: a linear power converter, a switching power converter, a potentiostat, a resistor, a capacitor, an inductor, a discrete semiconductor element, a diodes, a transistors, a microchip, an amplifier microchip, a programmable amplifier, an analogue switch, a multiplexer, a power stabiliser, a reference voltage source, an analogue-to-digital converter (ADCs), an digital-to-analogue modulator (DAC), a comparator, a digital timer, a timing component, a microcontroller, a central processing unit (CPU), a processor, a conversion scheme, a comparison schemes, a resistive divider, an analogue signal digitiser, a quartz resonator, a real-time clock, a timer, a level shifter, or a transceivers.

### Methods of Calibrating a Plurality of Gas Sensors

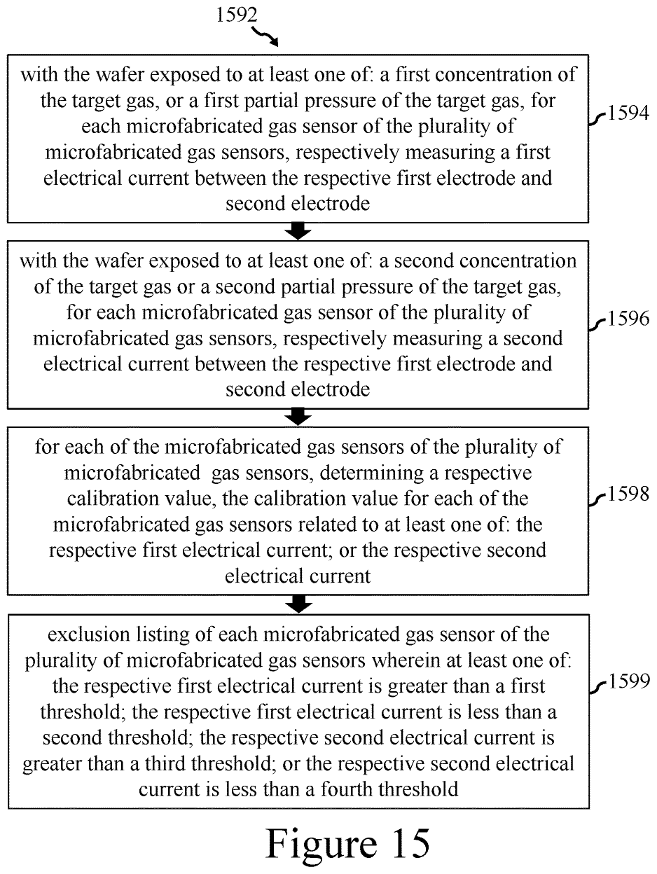

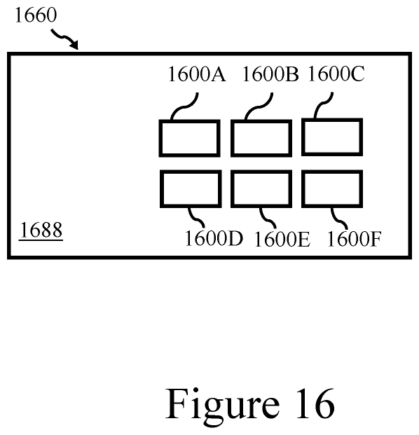

A description of examples herein relating to a method of calibrating a plurality of gas sensors is now given in relation to FIGS. 15 and 16. The gas sensors are as described herein. In some examples, such as those of FIGS. 15 and 16 the plurality of gas sensors are microfabricated.

In the examples of FIGS. 15 and 16 the plurality of gas sensors are microfabricated. In some other examples the plurality of gas sensors are not microfabricated.

FIG. 15 is a flow diagram of an example method of calibrating a plurality of microfabricated gas sensors. FIG. 16 shows schematically respective side cross-sections of the gas sensor during the method of FIG. 4.

Where a feature in relation to FIG. 16 corresponds with a feature described using FIG. 12, a reference numeral is used which is 400 greater than the corresponding reference numeral used for FIG. 12 (e.g., 1262 in FIG. 12 is 1662 in FIG. 16); corresponding descriptions for such features apply here also.

The wafer 1688 supports a plurality of microfabricated gas sensors 1600A, 1600B, 1600C, 1600D, 1600E, 1600F as described herein. The method 1592 of calibrating the plurality of microfabricated gas sensors comprises: with the wafer 1688 exposed to at least one of a first concentration of the target gas, or a first partial pressure of the target gas, for each microfabricated gas sensor of the plurality of microfabricated gas sensors 1600A, 1600B, 1600C, 1600D, 1600E, 1600F, respectively measuring 1594 a first electrical current between the respective first electrode and second electrode. The concentration of the target gas and/or a partial pressure of the target gas is, for example provided by a gas chamber. In some such examples, the temperature and the humidity of the wafer 1688 are also controlled. The method 1592 then comprises with the wafer 1688 exposed to at least one of a second concentration of the target gas, or a second partial pressure of the target gas, for each microfabricated gas sensor of the plurality of microfabricated gas sensors 1600A, 1600B, 1600C, 1600D, 1600E, 1600F, respectively measuring 1596 a second electrical current between the respective first electrode and second electrode. Next, the method 1592 comprises for each of the microfabricated gas sensors of the plurality of microfabricated gas sensors 1600A, 1600B, 1600C, 1600D, 1600E, 1600F, determining 1598 a respective calibration value, the calibration value for each of the gas sensors related to at least one of: the respective first electrical current; or the respective second electrical current. Then, the method 1592 comprises exclusion listing of each microfabricated gas sensor of the plurality of microfabricated gas sensors where at least one of: the respective first electrical current is greater than a first threshold; the respective first electrical current is less than a second threshold; the respective second electrical current is greater than a third threshold; or the respective second electrical current is less than a fourth threshold.

In some examples, determining the calibration value comprises determining the calibration curve for the target gas for each microfabricated gas sensor of the plurality of microfabricated gas sensors. The calibration curve for a microfabricated gas sensor is a relationship between the concentration of the target gas and the electrical current between the first electrode and the second electrode and is, for example determined over a desired range of the concentrations of the target gas which the gas sensor is desired to function within. The calibration value or the calibration curve for each gas sensor is, for example, related to at least one of: the respective first electrical current; or the respective second electrical current.

In some examples, the plurality of microfabricated gas sensors comprises no fewer than at least one of 100 gas sensors, 1000 gas sensors, or 10000 gas sensors. The wafer is, for example an 8-inch diameter disc supporting from 1000 gas sensors to 100000 gas sensors.

In some examples, the method of calibration is no more than 30 minutes long, or no more than 10 minutes long.

In some examples, the calibration value for each microfabricated gas sensor of the plurality of microfabricated gas sensors or the calibration curve for each microfabricated gas sensor of the plurality of microfabricated gas sensors is stored as a map of values for the wafer. In some examples the map is recorded in a microcontroller used for readout electronics for the calibration method. Alternatively, the calibration curve and the measurement data can be matched in the cloud to get an accurate reading.

In some examples, the method of calibration comprises electrical impedance spectroscopy (EIS) of each microfabricated gas sensor of the plurality of microfabricated gas sensors. In some such examples, the method of calibration comprises: (i) applying an alternating potential difference between the first electrode and the second electrode of each microfabricated gas sensor of the plurality of microfabricated gas sensors; and (ii) measuring an electrical current between the first electrode and the second electrode of each microfabricated gas sensor of the plurality of microfabricated gas sensors. In some such examples, (i) comprises applying a sinusoidal potential difference between the first electrode and the second electrode. The electrical current between the first electrode and the second electrode may have a different amplitude and/or phase than the sinusoidal potential difference between the first electrode and the second electrode. By varying the frequency of the alternating potential difference, the impedance between the first electrode and the second electrode can be measured at each frequency of the alternating potential difference for each microfabricated gas sensor of the plurality of microfabricated gas sensors. In some examples, the spectrum of frequency of the alternating potential difference against impedance is used to determine a property of each microfabricated gas sensor of the plurality of microfabricated gas sensors. In some such the examples the impedance spectroscopy is used to determine the resistivity of the electrolyte of each microfabricated gas sensor of the plurality of microfabricated gas sensors.

In some examples, at least one of the first threshold, the second threshold, the third threshold, or the fourth threshold is calculated by at least one of: emulating future usage, using accelerated aging protocols, or testing a gas sensor of the plurality of gas sensors at an elevated humidity and/or temperatures to simulate future use.

In some examples, the method of calibration comprises generating a digital twin of each microfabricated gas sensor of the plurality of microfabricated gas sensors using the respective calibration values of the plurality of gas sensors. In some such examples, the method of calibration comprises using the digital twin of each microfabricated gas sensor to predict the degradation and/or aging of each microfabricated gas sensor of the plurality of microfabricated gas sensors. Further, prediction of the degradation and/or aging of each microfabricated gas sensor of the plurality of microfabricated gas sensors, in some examples, allows recalibration of each microfabricated gas sensor of the plurality of microfabricated gas sensors as it ages and/or degrades.

### Uses of Gas Sensors

A description of examples herein relating to uses of the gas sensors described herein is now given.

In some examples, the gas sensor is used to monitor a concentration of at least one of: oxygen gas, carbon dioxide gas, carbon monoxide gas, hydrogen gas, or a fuel.

Some examples herein relate to uses of the gas sensor described herein to at least one of: (i) monitor food packed, transported, and/or stored in nitrogen atmosphere; (ii) monitor the exhaled breath of an individual in a wearable device, handheld device, or part of a larger instrument and/or medical device; (iii) to monitor the fitness or biometrics of, for example, a user in, for example a wearable fitness device or handheld monitors for industrial and/or safety applications; (iv) monitor indoor farming, for example, to monitor plant metabolism in an indoor environment; (v) monitor hydrogen storage and leakages; (vi) monitor dissolved oxygen in water, for example, to ensure aquatic creatures can survive in captivity, analyse microorganisms in a bioreactor, grow plants in aquaponics setups, or for blood analysis; (vii) monitor the gas mixture from a combustion chamber/engine, such as a hydrogen internal combustion engine; or (viii) monitor fuel storage, the operation of an electrolyser, fuel-cell, or other electrochemical device; (ix) to monitor battery off-gassing; or (x) monitor gases in transformer oil.

Some examples herein relate to the uses of the gas sensor described herein to at least one of: monitor a partial pressure of the target gas at the gas sensor, or monitor a flow of a fluid (e.g. a liquid or a gas). Examples of such uses include, for example: (i) monitor flow chemistry; (ii) monitor pressurised vessels; (iii) monitor weather; (iv) monitor an environment; (v) monitor a tyre pressure; (vi) monitor food packed, transported, and/or stored in nitrogen atmosphere; (vii) monitor the exhaled breath of an individual in a wearable device, handheld device, or part of a larger instrument and/or medical device; (viii) monitor the fitness or biometrics of, for example, a user in, for example a wearable fitness device or handheld monitors for industrial and/or safety applications; (ix) monitor indoor farming, for example, to monitor plant metabolism in an indoor environment; (x) monitor hydrogen storage and leakages; (xi) monitor the gas mixture from a combustion chamber/engine, such as a hydrogen internal combustion engine; or (xii) monitor fuel storage, the operation of an electrolyser, for electrolysis, for an electrolyser, fuel-cell, or other electrochemical device; (xiii) to monitor battery off-gassing; (xiv) monitor gases in transformer oil; (xv) for oven pyrolysis; (xvi) for a switchgear; or (xvii) for air quality measurement.

In some examples, the gas sensor is combined with another sensors, either by being placed next to each other, within the same package, or on the same die, for example, a MEMS-type sensor or another gas sensors. Further, some examples herein relate to uses of the gas sensor described herein to monitor gas concentrations in a natural gas pipe, and/or the use of the gas sensor described herein as a disposable component/in a cartridge of a larger device analysing oil, breath, or blood.

### Chemistry of the Electrolyte, Precursor and Polymer

A description of examples of the polymer, precursor, and electrolyte referred to herein is now given with reference to FIGS. 17 to 25, to elaborate on features of examples described herein.

In some examples, the electrolyte comprises a polymer comprising: a silicone, an anion and a cation, at least one of the cation or the anion covalently bonded to the silicone. Such examples FIGS. 17 to 25 schematically show structures of polymers in accordance with examples. In other examples, the polymer does not comprise a silicone and comprises at least one of an organic polymer, and inorganic polymer, polyethylene, polypropylene, polyurethane, polyester, polytetrafluoroethylene, polyamide, or polyimide.

FIGS. 17 to 21 each schematically show classes of polymers of examples with a cation or anion bonded to the silicone. Other classes are envisaged.

FIG. 17 schematically shows an example polymer and cation, or anion bonded to the silicone (R10).

Examples cations that could be bonded to the silicone include: triazolium, thioimidazolium, a quaternary ammonium, a protonated tertiary amine, imidazolium, substituted imidazolium, a quaternary phosphonium, a protonated tertiary phosphine, chloronium, pyrrolidinium, a substituted pyrrolidinium, pyridinium, a substituted pyridinium, a sulfonium or a substituted sulfonium.

Examples anions that could be bonded to the silicone include: a sulfonate, a phosphonate, alkoxide, a triflate, a thiolate, an imidazolate, tosylate, (methyl)acrylate, carboxylate, acetate, dimethylphosphate, or methylsulfate. Other cations and anions are envisaged, for example: tetrakis(pentafluorophenyl) borate, bis(fluorosulfonyl)imide, hexafluorophosphate, bis(trifluoromethane) sulfonimide (bistriflimide/TFSI)), tetrafluoroborate, chloride, bromide, or iodide.

R20 is a linker or a covalent bond; in further examples it is envisaged that molecular variants of such cations or anions are covalently bonded to the polymer. Example linkers include ethane-1,2-diyl, 1-oxopropane-1,3-diyl, 1-oxoethane-1,2-diyl and 1,5-dioxo-3-hydroxy-pentane-1,5-diyl, illustrated below:

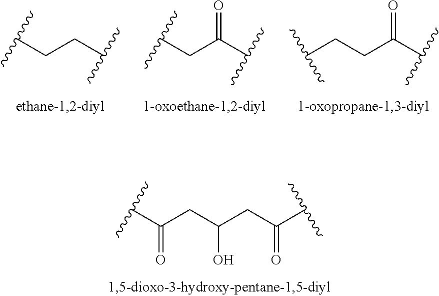

though different linkers are envisaged in further examples. The choice of the properties of the linker, such as length, allow the tuning of the conductivity and morphology of any ion channels in the electrolyte.

In some examples the anion or the cation are: a zwitterion, a multi-cation, or a multi-anion (e.g. cationic functionalised imidazolium such as butyl-methyl-imidazol-2-ylidene borane, others are envisaged) which may allow different ion channel structures in the electrolyte and modification of the ionic conductivity.

A person skilled in the art will appreciate that the terms n, m, and o in FIGS. 17 to 25 indicate the number of repeating units of each polymeric block. Each term could be, for example, any integer between 1 and 100,000 (including 1). In some examples a plurality of the terms are equal.

An example of a polymer comprising a silicone and cation or anion is schematically illustrated by FIG. 18. The silicone is a random copolymer. R30 is the cation or anion. The same examples are envisaged for R30 as outlined for R10. R40 is a linker or a covalent bond, with the same examples envisaged for R40 as outlined for R20. R50 is a crosslinker to at least one of another silicone or a second polymer. In some examples the crosslinker has been crosslinked by a catalytic reaction, for example, a photoinitiated alkyne-azide click reaction.

A slash symbol “/” through a bond is used in FIGS. 18 to 25 to indicate separate subunits of the copolymer and the random nature of the copolymer, that is, that the sequence and connectivity as well as the stoichiometry of the two or more subunits is variable. Alternatively, the copolymers are, for example, a dendrimers, and/or a copolymers of other configurations such as block, diblock, triblock, alternate, and/or graft copolymers.

The polymer being crosslinked allows modification of the physical and/or chemical properties of the electrolyte. In some examples, crosslinking makes the electrolyte solid state. In some examples the photolithographic formation of the electrolyte comprises crosslinking of the polymer.

An example of a polymer comprising a silicone and cation or anion is schematically illustrated by FIG. 19. The silicone is a random copolymer. R60 is the cation or anion. The same examples are envisaged for R60 as outlined for R10. R70 is a linker or a covalent bond; the same examples are envisaged for R70 as outlined for R20. In some examples, the chosen stoichiometry of the two subunits allows the tuning of the conductivity and morphology of any ion channels of the electrolyte.

An example of a polymer comprising a silicone and cation or anion is schematically illustrated by FIG. 20. The silicone is a block copolymer. R80 is the cation or anion. The same examples are envisaged for R80 as outlined for R10. R90 is a linker or a covalent bond, with the same examples envisaged for R90 as outlined for R20. In some examples, the choice of values of n, m and o allows the tuning of the conductivity and morphology of any ion channels of the electrolyte. In some examples, m is zero.

An example of a silicone and cation or anion is schematically illustrated by FIG. 21. R110 is the cation or anion. The same examples are envisaged for R110 as outlined for R10. R120 is a linker or a covalent bond; the same examples are envisaged for R120 as outlined for R20. R100 is a crosslinker; the same examples are envisaged for R100 as outlined for R50.

In some examples, the polymer is a first polymer and the electrolyte comprises a second polymer, for example a polystyrene, a polyacrylate or a second silicone. In some examples the mass ratio of the first polymer to the second polymer is between 1:100 and 100:1, in further examples the mass ratio of the first polymer to the second polymer is between 1:5 and 5:1. The second polymer in some examples comprises siloxane, and in other examples the second polymer is a non-siloxane polymer (in other words a polymer without siloxane). In some examples, when the second polymer is crosslinked, the first polymer is reduced in mobility, e.g. immobilised and/or the first polymer cannot be removed from the crosslinked second polymer by being dissolved in a solvent. In other examples, when the second polymer is crosslinked, the first polymer is not immobilised and/or the first polymer can be removed from the crosslinked second polymer by being dissolved in a solvent.

Some examples of an electrolyte of the present disclosure, the electrolyte contains a plurality of different polymers, for example, each having the general formula of one of FIGS. 17 to 21, for example each in accordance with the general formula of FIG. 17. In other examples the different polymers are each of a different general formula. The degree to which each component of a mixture comprising a single polymer species having the general formula can be achieved is limited by the purity of available starting materials, the synthetic routes used to make the compositions, and the degree of purification that can be achieved.

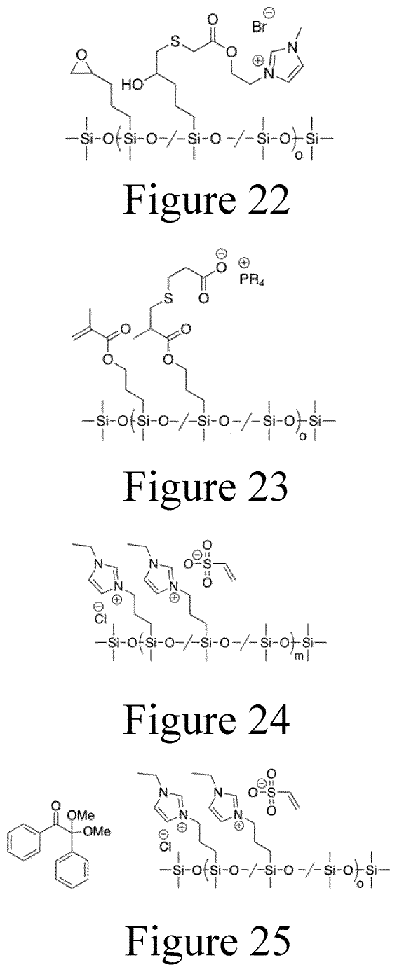

FIGS. 22 and 23 each schematically show example polymers as described herein. Other polymers are envisaged.

The polymer of FIG. 22 comprises a random copolymer, an anion bonded to the silicone, and a cation. The polymer, in some examples, is synthesised by combining 1-methyl-3-ethyl sulfanyl-acetyl-imidizolium bromide and poly-1,2-epoxypentyl-siloxane-random-siloxane at a 1:2 thiol:epoxy ratio. This mixture is solubilised with isopropanol and the reaction catalysed with 1 mol % (a person skilled in the art will appreciate the use of mole percent) diisopropylethylamine. After stirring the mixture for 16 hours at room temperature (298 Kelvin), the random copolymer is isolated by precipitation in diethylether and evaporation of volatiles. An electrolyte, may be produced by depositing and crosslinking a mixture produced by mixing the polymer of FIG. 22 with a 5 wt % (4-methylthiophenyl)methyl phenyl sulfonium trifluoromethanesulfonate. The mixture is, for example, deposited on a substrate by spin-coating the mixture on to a substrate at 3000 revolutions per minute (RPM, equivalent to 60× s-1 in S.I. units) for one minute. A region of the mixture on the substrate is crosslinked by selectively exposing the region of the mixture on the substrate to ultraviolet (UV) radiation (for example, from a mercury bulb through a photomask; however, other UV sources such as a light emitting diodes or lasers are envisaged) for 30 seconds. The substrate is then heated by being placed in contact with a hot plate at 338K for two minutes followed by 368K for two minutes. The region of the mixture that has not been crosslinked by the UV radiation is dissolved and removed using acetone, which produces a patterned crosslinked material e.g. an electrolyte on the substrate.

FIG. 23 schematically illustrates an example polymer as described above. The polymer, in some examples, is synthesised by combining poly-propyl-acetyl-siloxane-random siloxane and phosphonium 3-sulfanylpropanoic acid at a 1:2 thiol: acrylate ratio. This mixture is solubilised with isopropanol and heated to 323 Kelvin. After stirring for 16 hours at room temperature (298 Kelvin), the mixture is precipitated in diethylether and the volatiles are evaporated to isolate the polymer.

An electrolyte, in some examples, is produced by depositing and crosslinking a mixture produced by mixing the polymer of FIG. 23 with 10 wt % pentaerythritol tetraacrylate and 5 wt % 2,2-dimethoxy-1,2-diphenylethanone. This mixture is e.g. deposited on the substrate by spin coating the mixture on to a substrate at 3000 RPM for one minute. A region of the mixture on the substrate is crosslinked by exposing the region to UV light through a photomask for 30 seconds. The region of the mixture that has not been crosslinked by the UV radiation is dissolved and removed using isopropanol.

FIG. 24 illustrates a polymer of further examples. The polymer is a random copolymer and, in some examples, is synthesised by combining substituted imidazole (1-ethyl-imidizole) and 1-chloro-propyl-siloxane-random-siloxane at a 2:1 imidazole: alkylchloride ratio. The mixture is solubilised with n-butanol and slowly heated to 353 Kelvin. After stirring for 16 h at room temperature (298 Kelvin), the mixture is precipitated in diethylether and the volatiles are evaporated to isolate a polymer. The silicone is derived from the polymer by mixing the polymer with a sub-stoichiometric amount of sodium vinylsulfonate.

An electrolyte, in some examples, is produced by depositing and crosslinking a mixture of the silicone of FIG. 35 and 1,2-bis(2-mercaptoethoxy) ethane at a 1:1 ratio of thiol to -ene and 2 wt % 2,2-dimethoxy-1,2-diphenylethanone. The mixture is deposited on a substrate by spin-coating the mixture on to the substrate at 3000 RPM for one minute. A region of the mixture on the substrate is crosslinked by is exposing the region to UV radiation through a photomask for 30 seconds. The region of the mixture that has not been crosslinked by the UV radiation is dissolved and removed using acetone. In other examples, the addition of 1,2-bis(2-mercaptoethoxy) ethane is forgone as the silicone already comprises a crosslinker.

FIG. 25 illustrates example components of the electrolyte, specifically a photo-initiator (2,2-dimethoxy-1,2-diphenylethanone), an example polymer with a substituted imidazolium covalently bonded to a siloxane backbone, and crosslinker (2,2′-(ethylenedioxy) diethanethiol). The photoinitiator is used to facilitate the crosslinking. A person skilled in the art will appreciate that under the influence of light the 2,2-dimethoxy-1,2-diphenylethanone will form radicals which initiate the radical polymerisation; however, other photoinitiators and mechanisms of photoinitiated polymerisation are envisaged. For some examples a photoinitiator is not needed due to the choice of radiation and/or the choice of crosslinking chemistry.

A description of examples of the precursor used herein is now given, to elaborate on features of examples described herein.

In some examples the precursor comprises a monomer, for example, 1,6-hexanedioldiacrylate, though different monomers are envisaged in further examples. As will be appreciated, a monomer can be considered a molecule which undergoes a polymerisation reaction to form a polymer chain. In examples the monomer is polymerised to form a polymer, for example poly(1,6-hexanedioldiacrylate), though other polymers are envisaged in other examples. In some examples the silicone bonded to at least one of the cationic component or the anionic component has reduced mobility (e.g. is immobilised and/or cannot be removed from the mixture by being dissolved in a solvent), because it is entangled or otherwise immobilised with the polymerised monomer.

In some examples the precursor comprises a polymerisable and crosslinkable monomer, for example, SU-8, illustrated below:

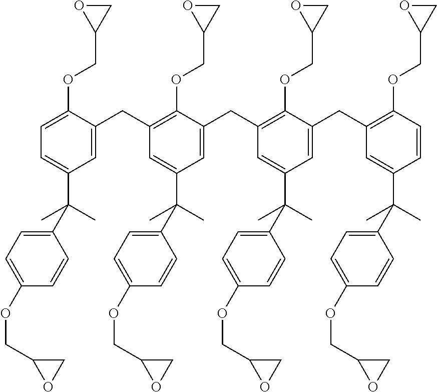 [SMILES: CC(C)(c1ccc(OCC2CO2)cc1)c1ccc(OCC2CO2)c(Cc2cc(C(C)(C)c3ccc(OCC4CO4)cc3)cc(Cc3cc(C(C)(C)c4ccc(OCC5CO5)cc4)cc(Cc4cc(C(C)(C)c5ccc(OCC6CO6)cc5)ccc4OCC4CO4)c3OCC3CO3)c2OCC2CO2)c1]

different monomers are envisaged in further examples.

In some examples, the precursor comprises several different monomers which are polymerisable and crosslinkable. For example, the mixture comprises a first formulation comprising 50 wt % (a person skilled in the art will appreciate the use of weight percent) trimethyloylpropane triacrylate, 20 wt % 1,6-hexanediol diacrylate, 25 wt % acrylic acid, and a 5 wt % photoinitiator; trimethyloylpropane triacrylate 1,6-hexanediol diacrylate and acrylic acid are illustrated below.

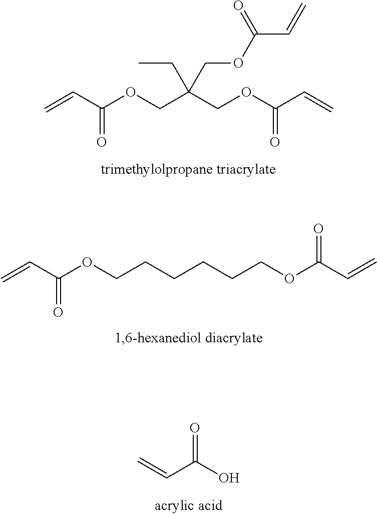

The first formulation is mixed with a second formulation comprising the polymer. A further example of a first formulation is a 50 wt % polyurethane acrylate and 45 wt % trimethylolpropane triacrylate and 5 wt % photoinitiator. In some examples the mass ratio of the first formulation to the second formulation is between 1:100 and 100:1, in further examples the mass ratio of the first formulation to the second formulation is between 1:5 and 5:1.

In some examples the precursor or the silicone comprises a crosslinker, for example: a polyester functionalised with an acrylate, a urethane functionalised with an acrylate, or an olefin; other polymers comprising a crosslinker envisaged. A person skilled in the art will appreciate that in some examples the crosslinker requires a catalyst, a photoinitiator and/or sensitizer to be crosslinked.

In examples, the precursor is crosslinkable or polymerisable mixture and is for manufacturing an electrolyte and comprises an additive or additives to modify the properties of the mixture or the mixtures when it has been crosslinked. In some examples additives such as butyl acrylate are added to the mixture to modulate the density of crosslinks when the mixture is crosslinked and/or plasticisers such as triethyleneglycol methylether methacrylate are added to plasticise the precursor and/or the electrolyte. In some examples the plasticiser is a reactive plasticiser, for example butyl acrylate, triethyleneglycol acrylate or siloxane acrylate. In other examples the plasticiser is a non-reactive plasticiser, for example propylene glycol or 1-ethyl-3-butyl-imidazolium tetrafluoroborate. Other plasticisers are envisaged. Changing the density of crosslinks or plasticising the mixture allows modification of the physical properties of the crosslinked mixture. Some examples of the precursor comprise a catalyst, a photoinitiator and/or sensitiser to allow and/or assist the crosslinking and/or polymerisation of the precursor.

Some examples allow the modification of the electronic and/or ionic properties of the precursor or of the electrolyte by the addition of a redox active additive, e.g. vinyl ferrocene. This allows the tuning of the electronic and/or ionic properties, for example for uses in the gas sensor.

In examples, the precursor comprises one or more additive to modify one or more properties of the mixture or resulting crosslinked/polymerised electrolyte. Such properties include for example electronic, catalytic, mechanical, optical, mass transport or processing properties. Such an additive is for example metallic, semiconducting, insulating, or non-metallic, and/or comprises nanoparticles, microparticles, clusters, fillers, nanotubes, a clay, graphene, quantum dots, and/or carbon such as carbon black or graphite. Such an additive, e.g. fillers, is present in the range of for example 0.0001 wt % to 98 wt %, such as 0.01 wt % to 30 wt %.

In some examples the precursor for manufacturing an electrolyte comprises a chromophore. For some examples a chromophore will modulate electromagnetic radiation applied to the mixture, which in some examples allows more efficient crosslinking. In examples, a chromophore is used to provide fluorescent properties to the at least partly crosslinked electrolyte. The chromophore is, for example, a rhodamine or a rhodamine derivative; however other chromophores are envisaged depending on the desired electromagnetic properties.

Many different examples of the precursor or the electrolyte are envisaged that possess ionic functionality and/or crosslinking functionality, as polymers with a variety of functional groups for crosslinking or suitable for attachment of a component of a salt are commercially available. Many different examples of salts and crosslinkers are envisaged as many suitable salts and crosslinkers are commercially available. In some examples, the crosslinker in the mixture is not bonded to the silicone. In some examples, the crosslinker is bonded to the silicone, allowing the silicone to crosslink with itself. A person skilled in the art will also appreciate that any of the silicones referred to herein can be, for example, a simple polymer, a dendrimer, and/or a copolymer of various configurations such as block, diblock, triblock, random, alternate, and/or graft copolymers.

In some examples the precursor comprises a plurality of different crosslinkers, in some examples the different crosslinkers are crosslinkable by different reaction pathways. In some examples both the silicone and a polymer in the precursor are crosslinkable. For example, the precursor comprises, a polyurethane acrylate and a combination of photoinitiator, other such mixtures are envisaged. In some examples of such mixtures a plurality of crosslinking mechanisms occur.

In some examples the precursor comprises a silicone where a first crosslinker is covalently bonded to the silicone; and a second crosslinker. For example, the precursor comprises SU-8, other examples are envisaged. In such examples, upon irradiation of this mixture a plurality of crosslinking mechanisms are envisaged. In some examples the mass ratio of the polymer to the monomer is between 1:100 and 100:1, in further examples the mass ratio of the polymer to the monomer is between 1:5 and 5:1.

In some examples, the electrolyte comprises a surfactant. The surfactant is, for example, non-ionic (e.g. polysorbate), anionic (e.g. sodium lauryl sulfate), cationic (e.g. benzalkonium chloride), or amphoteric (e.g. cocoamidpropl betaine). The surfactant allows the modification of structure of ion channels that are produced within the crosslinked electrolyte in some examples, which consequently allows tuning of the ion conductivity.

In some such examples, at least one of the anion or the cation comprises at least one of: a carbon-carbon covalent bond, or a carbon-hydrogen covalent bond.

In some examples, the cation and the anion when isolated in combination form an ionic liquid. An ionic liquid referred to herein is for example, when isolated, a liquid or glass at 373 Kelvin.

In some examples, the polymer comprises a linker covalently bonded to the silicone and at least one of the cation or anion. In some examples, the polymer is cross-linked. In some examples, the polymer is a first polymer, and the electrolyte further comprises a second polymer, and where at least one of: the second polymer comprises a silicone; the second polymer comprises a non-silicone; or the second polymer is crosslinked.

In some examples, the cation comprises at least one of: zwitterion, a triazolium, a thioimidazolium, a quaternary ammonium, a protonated tertiary amine, imidazolium, a substituted imidazolium, a quaternary phosphonium, a protonated tertiary phosphine, chlorinium, pyrrolidinium, a substituted pyrrolidinium, pyridinium, a substituted pyridinium, sulfonium or a substituted sulfonium.

In some examples, the anion comprises at least one of: zwitterion, a multi-anion, a sulfonate, a phosphonate, alkoxide, thiolate, an imidazolate, a tetrakis(pentafluorophenyl) borate, bis(fluorosulfonyl)imide, a tosylate, (methyl)acrylate, a carboxylate, acetate, chloride, bromide, iodide, dimethylphosphate, methylsulfate, hexafluorophosphate, a triflate, bis(trifluoromethane) sulfonimide (bistriflimide/TFSI)), or tetrafluoroborate.

In some examples, the electrolyte comprises at least one of: an additive for modification of crosslink density, a plasticiser, a redox active additive, a chromophore, a multi-cation.

In some examples, at least one of the electrolyte, the precursor, or the polymer is inorganic. Inorganic herein is not comprising a carbon atom bonded to at least one of: a hydrogen, oxygen, nitrogen, sulfur, or a halogen. In other examples, at least one of the electrolyte, the precursor or the polymer does not comprise a carbon-carbon covalent bond or a carbon-hydrogen covalent bond.

In some examples, the electrolyte is cross-linked. In some such examples, the electrolyte is insoluble.

In some examples, at least one of the electrolyte, the precursor, or the polymer is organic. Organic herein is comprising a carbon atom bonded to at least one of: a hydrogen, oxygen, nitrogen, sulfur, or a halogen. In some examples at least one of the electrolyte, the precursor or the polymer comprises at least one of a carbon-carbon covalent bond or a carbon-hydrogen covalent bond.

In some examples, at least one of the electrolyte, or the precursor is polymeric material or a ceramic material. A polymeric material herein comprises a polymer. A ceramic material herein comprises an inorganic material that is: a metal oxide, a metal nitride, a metal carbide, or silicon carbide.

### Further Description of Features

A description of some terms and features used herein is now given, to elaborate on features of examples described herein.

An electrolyte herein is a conductor of ions. In some examples, the ion conductivity of the electrolyte 102 at 20 Celsius (293 Kelvin) is: from 10 Mircosiemens per metre (μS/m) to 1 Millisiemens per metre (mS/m); or from 20 Mircosiemens (μS/m) per metre to 500 Mircosiemens per metre (μS/m). The ion conductivity of the electrolyte is, for example 400 Mircosiemens per metre (μS/m). A Siemens per metre (S/m) is equal to an Ampere squared second cubed per kilogram per metre cubed (kg-1·m-3·s3·A2). In some examples, the ion resistivity of the electrolyte herein at 20 Celsius (293 Kelvin) is from 1 Kiloohm metres (kΩ·m) to 100 Kiloohm metres (kΩ·m); or from 2 Kiloohm metres (kΩ·m) to 500 Kiloohm metres (kΩ·m). A person skilled in the art will appreciate that the direct current ion conductivity values obtained will depend on several variables, including the chosen composition of the electrolyte, any chosen additives, and the temperature of operation.

An active area of the electrolyte herein is the surface area of the electrolyte exposed to the target gas when the gas sensor is in use. In some examples, the active area is from 5 micrometres squared (μm2) to 1,000,000 micrometres squared (μm2), or from 113 micrometres squared (μm2) to 11,304 micrometres squared (μm2). The thickness of the electrolyte herein is, for example, from 100 nanometres (nm) to 50 micrometres (μm), or from 10 micrometres (μm) to 30 micrometres (μm).

A partial pressure of a target gas herein can be considered as the pressure exerted by the target gas. In some examples the target gas is in a mixture of gasses and the partial pressure of the target gas is a component of the total pressure exerted by the mixture of gasses as a whole. In other examples, the target gas is not in a mixture of gasses so the partial pressure is the pressure of the gas as a whole.

In some examples the gas sensor is a microfabricated, at least partly microfabricated, manufactured by microfabrication techniques, and/or manufactured at least partly by microfabrication techniques. Further, in some examples, the method of manufacturing a gas sensor is a method of microfabricating a gas sensor, and/or the plurality of gas sensors are microfabricated. Microfabrication herein is a method of manufacture comprising a process of creating structures with a dimension less than one millimetre. In some examples microfabrication comprises using microfabrication techniques, such as lithography, thin film deposition, etching, bonding, and/or patterning.

In some examples, the gas sensor herein is at least one of: micromachined, microelectronic, a semiconductor gas sensor, or part of an integrated circuit.

Solid-state herein is at least one of: solid, semi-solid, quasi-solid, substantially solid, predominantly solid, more solid than liquid (homogeneously throughout the material), a gel, or a glass. In some examples a solid contains no or substantially no (within functional tolerances) leachable free liquid or solvent.

A substrate may also be referred to as a chip, a slice, a wafer, a die or a layer. A substrate is, e.g., a generally planar or relatively thin portion of material, and in some examples is crystalline. A substrate may be a disc or part of a disc of crystalline Si for use in a semiconductor fabrication plant, and in some such examples is a 125 gram, 300 millimetre diameter disc. In some such examples the substrate is a 25 millimetre, 51 millimetre, 76 millimetre, 100 millimetre, 200 millimetre or 300 millimetre diameter disc. A substrate referred to herein is, for example, a single layer of the same homogenous material, though it is envisaged for other examples that a substrate instead comprises one or more layers or portions each deposited or formed independently of each other (for example one after another during a manufacture process to form a stack of sub-layers which together could be considered a substrate). In some examples, a substrate comprises portions of different materials, for example, for fabrication. In some examples, the substrate herein is a semiconductor, a polymer, and/or a dielectric. In some examples, the substrate comprises at least one of: glass, quartz, sapphire, a ceramic, a metal, a semi-metal, a polymer, carbon (C), silicon (Si), gallium (Ga), germanium (Gr), lithium niobate (LiNbO3), graphene (C), indium (In), or an alloy, oxide, nitride, or phosphide of at least one of such. In some examples the substrate is an electrical conductor.

A cap substrate herein is a substrate for bonding, attaching and/or connecting a different substrate. In some examples the cap substrate is an electrical conductor.

An electrical conductor, for example, comprises at least one of: a metal, a semiconductor, a dielectric, an n-type semiconductor, a metalloid, silicon (Si), gallium (Ga), germanium (Gr), lithium niobate (LiNbO3), graphene (C), indium, or an alloy, oxide, nitride, or phosphide of at least one of such. An electrical conductor comprises an electrically conductive material and may comprise an electrically conductive structure. The electrical conductivity of an electrical conductor at 20 Celsius (293 Kelvin) may be no less than at least one of 1 Siemens per metre, 10 Siemens per metre, 100 Siemens per metre, 1000 Siemens per metre, 10000 Siemens per metre, or 100000 Siemens per metre. The electrical resistivity of the layer at 20 Celsius (293 Kelvin) may be no more than at least one of 0.00001 Ohm metres, 0.0001 Ohm metres, 0.001 Ohm metres, 0.01 Ohm metres, 0.1 Ohm metres, or 1 Ohm metres. An Ohm metre (2/m) is equal to a kilogram metre cubed per Ampere squared per second cubed (kg·m3·s-3·A-2).

In some examples, a layer or portion herein is a single layer of the same homogenous material, though it is envisaged for other examples that a layer instead comprises one or more sub-layers or portions each deposited or formed independently of each other (e.g., one after another during a fabrication process to form a stack of sub-layers which together could be considered a layer). A layer or portion may have sub-portions of different materials, for example, for fabrication. Sub-portions of a layer or portion may have different dopant concentrations.

In some examples the potential different applied between the first electrode and the second electrode is at least one of from 50 Volts (V) to −50 Volts (V), from 10 Volts (V) to −10 Volts (V), or from 5.0 Volts (V) to 0.1 Volts (V). A potential difference of the first electrode is, for example, at least one of: from 25 Volts (V) to −25 Volts (V), from 5.0 Volts (V) to −5.0 Volts (V), or from 0.0 Volts (V) to −2.0 Volts (V). A potential difference of the second electrode is, for example, at least one of: from 25 Volts (V) to −25 Volts (V), from 5.0 Volts (V) to −5.0 Volts (V), from 0.0 Volts (V) to 3.0 Volts (V), or from 0.0 Volts (V) to 1.0 Volt (V). A Volt (V) is equal to a kilogram metre squared per second cubed per Ampere (kg·m2·s-3·A-1).

In some examples, at least one of the first electrode or the second electrode is an active electrode.

In some examples, the surface of the first electrode and/or the second electrode is from 3 micrometres squared (μm2) to 785,000 micrometres squared (μm2); or from 78.5 micrometres squared (μm2) to 7,850 micrometres squared (μm2). In some examples a cross-section of at least one of the first electrode or the second electrode is circular, substantially circular, or quasi-circular. Other shapes of the cross-section are envisaged, for example an oval or a square. In some examples, a radius of the first electrode and/or the second electrode is from 1 micrometre (μm) to 500 micrometres (μm). In some such examples radius of the first electrode and/or the second electrode is from 5 micrometres (μm) to 50 micrometres (μm). In some examples a surface area of the counter electrode is at least one of from 1 times to 100 times greater than; from 5 times to 15 times greater than; or 10 times a surface are of the working electrode. In some examples a thickness of at least one of the first electrode or the second electrode is from 10 nanometres (nm) to 10 micrometres (μm), or from 100 nanometres (nm) to 500 nanometres (nm). In some examples at least one of the first electrode or the second electrode is no more than 1 micrometre thick. In some examples at least one of the first electrode or the second electrode is at least one of: formed at least partly by thin film deposition, a microelectrode, a thin film electrode, or microfabricated. In some examples, the power consumption of the gas sensor reduced compared to known gas sensors not described herein as a result of the electrodes being smaller in such examples and/or the electrical current between the first electrode and the second electrode being less than known gas sensors not described herein.

In some examples the first electrode comprises at least one of: silver (Ag) silver chloride (AgCl), gold (Au), platinum (Pt), palladium (Pd), copper (Cu), chromium (Cr), titanium (Ti), carbon (C), a carbon allotrope such as graphite, or a mixture or alloy thereof.

In some examples the second electrode comprises at least one of: silver (Ag) silver chloride (AgCl), gold (Au), platinum (Pt), palladium (Pd), copper (Cu), chromium (Cr), titanium (Ti), carbon (C), a carbon allotrope such as graphite, or a mixture or alloy thereof. In some examples the first electrode and the second electrode are formed of the same materials. In other examples the first electrode is formed of a different material or different materials to the second electrode.

In some examples the first electrode comprises a first active area. The first active area for at least one of: reduction of the target gas, oxidation of the target gas, reduction of the target ion, or oxidation of the target ion. In some examples, the electrolyte is in contact with all of the first active area. In some examples the first active area is the area of the first electrode in contact with the first surface. In some examples, a diameter of the first surface is from 1 micrometre (μm) to 2,000 micrometres (μm) greater than a diameter of the first active area.

In some examples the second electrode comprises a second active area. In some examples, the second active area for at least one of: reduction of the target gas, oxidation of the target gas, reduction of the target ion, oxidation of the target ion, reduction of the electrolyte, oxidation of the electrolyte, reduction of a redox additive, or oxidation of a redox additive. In some examples, the electrolyte is in contact with all of the second active area. In some examples the second active area is the area of the second electrode in contact with the second surface. In some examples, a diameter of the second surface is from 1 micrometre (μm) to 2,000 micrometres (μm) greater than a diameter of the second active area.

In some examples, the first electrode is a working electrode, and the second electrode is a counter electrode. In some examples, the gas sensor comprises at least one of: a plurality of working electrodes, a plurality of counter electrodes, or a plurality of reference electrodes. In some examples, the pluralities of working, counter and/or reference electrodes are respectively spaced from each other.

An electrical current herein is a flow of electrons.

An ion channel herein is a structure that allows the transport of ions across the electrolyte. Ion channels may be formed by various materials, e.g., polymers, ceramics, metals, semi-metals or liquids. Ion channels may have different shapes, sizes, and orientations. In some examples, ion channels affect at least one of: the ion conductivity, or the stability of the electrolyte.

A silicone herein is a polymer that comprises a silicon-oxygen backbone chain. In some examples the silicone has an organic group attached to each silicon atom. A silicone is, for example, poly-siloxane.

Tuning ion conductivity is also, for example, achievable by varying the amount of anion and cation in the electrolyte, which, in some examples influences the shape and extent of the phase separated regions and formed ion channels, and in some examples affects the glass transition.

Attached herein is at least one of directly attached or indirectly attached. Attached, for example, comprises attached by at least one of: a chemical bond, a fusion bond, gold thermocompression bond, a glass frit bond, an eutectic bond, a solder, an adhesive bond, an anodic bond, a mechanical adherence, or a chemical adherence.

Attaching herein is at least one of directly attaching or indirectly attaching. Attaching, for example, comprises at least one of: forming a chemical bond, fusion bonding, gold thermocompression, glass frit bonding, eutectic bonding, soldering, adhesive bonding, anodic bonding, using an adhesive to mechanically and/or chemically adhere, processing, photocuring, exposure to a catalyst, application of pressure, a flip-chip process, a pick-and-place process, or tape-automated bonding (TAB).

In some examples forming or providing herein comprises a manufacture process, e.g., using known techniques such as: epitaxy, metalorganic vapour-phase epitaxy (MOVPE), surface passivation, lithography, photolithography, ion implantation, etching, dry etching ion etching, wet etching, buffered oxide etching, plasma ashing, plasma etching, thermal treatment, annealing, thermal oxidation, chemical vapor deposition, atomic layer deposition, physical vapor deposition, molecular beam epitaxy, laser lift-off, electrochemical deposition, electroplating, chemical-mechanical polishing, wafer fusion, anodic bonding, laser initiated forward transfer (LIFT), printing, screen printing, 3D-printing, additive manufacturing, drop casting, dip coating, spray coating, roll coating, painting, doctor blading, imprint lithography, or adhesion.

A Faraday cage herein is an enclosure that can block electromagnetic fields from entering or exiting the enclosure or reduce the intensity of electromagnetic fields transmitted through the enclosure. In some examples, the Faraday cage is a continuous electrically conductive material or a mesh of such materials. In some examples, the Faraday cage protects the gas sensor from external electromagnetic interference and/or radio frequency interference. In some examples, the Faraday cage has different configurations depending on the application and the gas sensor. In some examples the Faraday cage is a partial Faraday cage. In some examples, the Faraday cage comprises an opening, such as a port for the first electrode and the second electrode.

In some examples of the device herein, the device comprises a protection layer. The protection layer, for example preventing or reducing the passage of moisture or other environmental factors to the components of the device. In some examples the protection layer is at least one of: a metallic enclosure, a compound, protective coating, ridged, flexible, electrically conductive, electrically insulative, monolithic, porous, comprising a plurality of layers, or comprising a plurality of chemical and/or mechanical elements.

In some examples of the device herein, the features of the device are electrically connected using soldering. Various other methods of electrically connecting the features of the device are envisaged, for example at least one of: soldering, welding, wire bonding, conductive adhesive, non-conductive adhesive, or spring contacts.

In some examples of the device herein, the features of the device are mounted on the surface of the board. Other methods of mounting are envisaged.

In some examples of the device herein the device comprises an integrated preamplifier and/or an integrated potentiostat. In some such example the impact of electromagnetic interference on the device is reduced, the accuracy of the gas sensor is increased, and/or the sensitivity of the gas sensor is increased.

It is to be understood that any feature described in relation to any one example may be used alone, or in combination with other features described, and may also be used in combination with one or more features of any other of the example, or any combination of any other of the examples. Furthermore, equivalents and modifications not described above may also be employed without departing from the scope of the accompanying claims.

### CLAUSES

Clause 1. A microfabricated gas sensor for a target gas, comprising: an electrolyte at least partly provided using photolithography; a first electrode in contact with a first surface of the electrolyte and for reducing or oxidising the target gas received through the electrolyte to produce target ions; and a second electrode in contact with a second surface of the electrolyte, wherein with a potential difference applied between the first and second electrodes an amount of electrical current between the first electrode and the second electrode is indicative of at least one of: a concentration of the target gas at the first surface, or a partial pressure of the target gas at the first surface.

Clause 2. The microfabricated gas sensor of claim 1, wherein the electrolyte is inorganic.

Clause 3. The microfabricated gas sensor of any previous claim, wherein the electrolyte is solid-state.

Clause 4. The microfabricated gas sensor of any previous claim, wherein the electrolyte is at least partly patterned by photolithography.

Clause 5. The microfabricated gas sensor of any previous claim, wherein the first surface of the electrolyte and the second surface of the electrolyte do not overlap and are spaced from each other.

Clause 6. The microfabricated gas sensor of any previous claim, comprising a substrate supporting the electrolyte, the first electrode, and the second electrode.

Clause 7. The microfabricated gas sensor of claim 6, wherein the substrate comprises at least one of: glass, quartz, sapphire, a ceramic, a metal, a semi-metal a polymer, or carbon.

Clause 8. The microfabricated gas sensor of claim 6 or claim 7, wherein providing the electrolyte using photolithography comprises: providing a precursor; providing a photomask on the precursor; and irradiating the precursor to form the electrolyte.

Clause 9. The microfabricated gas sensor of any of claims 6 to 8 comprising a cap, and a cavity between the cap and a sensing surface of the electrolyte, the cap comprising: a cap substrate attached to the substrate; and a gas channel from the cavity and through the cap substrate.

Clause 10. The microfabricated gas sensor of claim 9, comprising a gas-permeable layer on the cap substrate, the gas channel from the gas-permeable layer to the cavity.

Clause 11. The microfabricated gas sensor of claim 9 or claim 10, wherein the cap comprises an electrically conductive layer between the cap substrate and the cavity.

Clause 12. The microfabricated gas sensor of claim 11, wherein the electrically conductive layer and the substrate are electrically connected.

Clause 13. The microfabricated gas sensor of any of claims 9 to 12, wherein the substrate and the cap substrate are attached to each other by an electrically conductive portion.

Clause 14. The microfabricated gas sensor of any of claims 9 to 13, wherein the cap substrate is electrically conductive.

Clause 15. The microfabricated gas sensor of any previous claim, comprising a Faraday cage, the electrolyte, the first electrode, and the second electrode within the Faraday cage.

Clause 16. The microfabricated gas sensor of any previous claim, wherein the target gas is oxygen.

Clause 17. The microfabricated gas sensor of any previous claim, wherein the electrolyte comprises a polymer comprising: a silicone, an anion, and a cation, at least one of the cation or the anion covalently bonded to the silicone.

Clause 18. The microfabricated gas sensor of claim 17, wherein the polymer comprises a linker covalently bonded to the silicone and at least one of the cation or anion.

Clause 19. The microfabricated gas sensor of any of claim 17 or claim 18,
wherein at least one of the anion or the cation comprises at least one of: a carbon-carbon covalent bond, or a carbon-hydrogen covalent bond.

Clause 20. The microfabricated gas sensor of any previous claim, wherein the electrolyte is cross-linked.

Clause 21. The microfabricated gas sensor of any previous claim, wherein the first electrode is a working electrode, and the second electrode is a counter electrode.

Clause 22. The microfabricated gas sensor of any of claims 1 to 20, wherein the first electrode is a first working electrode and the microfabricated gas sensor comprises a second working electrode in contact with a third surface of the electrolyte, the third surface not overlapping with and spaced from the first surface and the second surface.

Clause 23. The microfabricated gas sensor of claim 23, wherein the target gas is a first target gas, the first working electrode is for sensing the first target gas, and the second working electrode is for sensing a second target gas different to the first target gas.

Clause 24. The microfabricated gas sensor of any previous claim, comprising at least one of: a reference electrode in contact with the electrolyte; a diagnostic electrode in contact with the electrolyte; or a grounding electrode in contact with the electrolyte.

Clause 25. The microfabricated gas sensor of any previous claim, wherein the polymer is a first polymer, and the electrolyte further comprises a second polymer, and wherein at least one of: the second polymer comprises a silicone; the second polymer comprises a non-silicone; or the second polymer is crosslinked.

Clause 26. The microfabricated gas sensor of any previous claim, wherein at least one of: the cation comprises at least one of: a triazolium, a thioimidazolium, a quaternary ammonium, a protonated tertiary amine, imidazolium, a substituted imidazolium, a quaternary phosphonium, a protonated tertiary phosphine, chlorinium, pyrrolidinium, a substituted pyrrolidinium, pyridinium, a substituted pyridinium, sulfonium or a substituted sulfonium; the anion comprises at least one of: a sulfonate, a phosphonate, alkoxide, a thiolate, an imidazolate, tetrakis(pentafluorophenyl) borate, bis(fluorosulfonyl)imide, a tosylate, (methyl)acrylate, a carboxylate, acetate, chloride, bromide, iodide, dimethylphosphate, methylsulfate, hexafluorophosphate, a triflate, bis(trifluoromethane) sulfonimide (bistriflimide/TFSI)), or tetrafluoroborate; the electrolyte comprises an additive for modification of crosslink density; the electrolyte comprises a plasticiser; the electrolyte comprises a redox active additive; the electrolyte comprises a chromophore; the electrolyte comprises a surfactant; the cation is a multi-cation; the anion is a multi-anion; the cation is a zwitterion; or the anion is a zwitterion.

Clause 27. The microfabricated gas sensor of any previous claim, wherein upon application of the potential difference between the first electrode and the second electrode, an electrical current between the first electrode and the second electrode is no more than 1 Milliampere.

Clause 28. The microfabricated gas sensor of any previous claim, configured to be used when at least partly submerged or submerged in a liquid to determine at least one of: the concentration of the target gas at the first surface, or the partial pressure of the target gas at the first surface.

Clause 29. The microfabricated gas sensor of any previous claim, configured to be used in a humidity from 0 percent to 100 percent to determine at least one of: the concentration of the target gas at the first surface, or the partial pressure of the target gas at the first surface.

Clause 30. The microfabricated gas sensor of any previous claim, configured to be used at a temperature at least one of: from 194 Kelvin to 573 Kelvin, from 220 Kelvin to 573 Kelvin, or from 220 Kelvin to 453 Kelvin to determine at least one of: the concentration of the target gas at the first surface, or the partial pressure of the target gas at the first surface. Clause 31. A method of microfabricating a gas sensor of any previous claim.

Clause 32. A method of microfabricating a gas sensor for a target gas, comprising: providing an electrolyte at least partly using photolithography; providing a first electrode in contact with a first surface of the electrolyte and for reducing or oxidising the target gas received through the electrolyte to produce target ions; and providing a second electrode in contact with a second surface of the electrolyte, wherein with a potential difference applied between the first and second electrodes an amount of electrical current between the first electrode and the second electrode is indicative of at least one of a concentration of the target gas at the first surface or a partial pressure of the first gas at the first surface.

Clause 33. The method of claim 32, wherein the electrolyte is inorganic.

Clause 34. The method of claim 32 or 33, wherein the electrolyte is solid-state.

Clause 35. The method of any of claims 32 to 34, wherein the first surface of the electrolyte and the second surface of the electrolyte do not overlap and are spaced from each other.

Clause 36. The method of any of claims 32 to 35, wherein the electrolyte is at least partly patterned by photolithography.

Clause 37. The method of any of claims 32 to 36, comprising providing a substrate for supporting the electrolyte, the first electrode, and the second electrode.

Clause 38. The method of claim 37, wherein the substrate comprises at least one of: glass, quartz, sapphire, a ceramic, a metal, a semi-metal, a polymer, or carbon.

Clause 39. The method of any of claim 37 or claim 38, comprising: providing a precursor on the substrate; and photolithography of the precursor to provide the electrolyte.

Clause 40. The method of claim 39, comprising: providing a photomask on the precursor; and irradiating the precursor.

Clause 41. The method of claim 39 or claim 40, wherein at least one of: the precursor comprises a photoinitiator; the precursor is a photoresist mixture; the precursor is a negative photoresist mixture; the cation comprises at least one of: a triazolium, a thioimidazolium, a quaternary ammonium, a protonated tertiary amine, imidazolium, a substituted imidazolium, a quaternary phosphonium, a protonated tertiary phosphine, chlorinium, pyrrolidinium, a substituted pyrrolidinium, pyridinium, a substituted pyridinium, a sulfonium or substituted sulfonium; the anion comprises at least one of: a sulfonate, a phosphonate, alkoxide, a thiolate, an imidazolate, tetrakis(pentafluorophenyl) borate, bis(fluorosulfonyl)imide, a tosylate, (methyl)acrylate, a carboxylate, acetate, chloride, bromide, iodide, 5 dimethylphosphate, methylsulfate, hexafluorophosphate, a triflate,bis(trifluoromethane) sulfonimide (bistriflimide/TFSI)), or tetrafluoroborate; the precursor comprises a plasticiser; the precursor comprises a redox active additive; the precursor comprises a chromophore; the precursor comprises a surfactant; the electrolyte comprises an additive for modification of crosslink density; the electrolyte comprises a plasticiser; the electrolyte comprises a redox active additive; the electrolyte comprises a chromophore; the electrolyte comprises a surfactant; the cation component comprises a multi-cation; the anion component comprises a multi-anion; the cation is a zwitterion; or the anion is a zwitterion.

Clause 42. The method of any of claims 39 to 41, wherein at least one of: i) the precursor comprises a crosslinker and the method comprises crosslinking a region of the precursor to form a region of crosslinked precursor and a region of precursor that has not been crosslinked; or ii) the precursor comprises a monomer and the method comprises polymerising a region of the precursor to form a region of polymerised precursor and a region of precursor that has not been polymerised.

Clause 43. The method of claim 42, comprising at least one of: etching away a region of the precursor that has been at least one of crosslinked or polymerised; exposing the precursor to radiation to cause at least one of the crosslinking or the polymerising; exposing the precursor to an electron beam or electromagnetic radiation to cause at least one of the crosslinking or the polymerising; exposing the precursor to electromagnetic radiation to cause at least one of the crosslinking or the polymerising; or heating the precursor to cause at least one of the crosslinking or the polymerising.

Clause 44. The method of claim 42 or claim 43, comprising at least one of: at least partly removing the region of the precursor that has not been crosslinked; at least partly removing the region of the precursor that has not been polymerised; at least partly dissolving the region of the precursor that has not been crosslinked; or at least partly dissolving the region of the precursor that has not been polymerised.

Clause 45. The method of any of claims 42 to 44, wherein at least one of the crosslinker or the monomer comprises at least one of: a vinyl group, an acrylate group, an azide, an alkyne, an epoxy, a thiol, an olefin, an alcohol, an aldehyde, a carboxylate, an amine, a phosphine, a diene, diethylene glycol divinyl ether or diethylene glycol diacrylate; or an additive for modification of crosslink density.

Clause 46. The method of any of claims 42 to 45, wherein the crosslinker is covalently bonded to the monomer.

Clause 47. The method of any of claims 39 to 46, comprising at least one of: depositing the precursor on the substrate; depositing the first electrode on the substrate; depositing the second electrode on the substrate; spin coating the precursor on the substrate; or printing the precursor on the substrate.

Clause 48. The method of any of claims 39 to 47, comprising a providing a cap, and providing a cavity between the cap and a sensing surface of the electrolyte.

Clause 49. The method of claim 48, wherein providing the cap comprises: providing a cap substrate; and attaching the cap substrate to the substrate to form the cavity.

Clause 50. The method of claim 48 or claim 49, comprising: providing a gas-permeable layer on the cap substrate; and providing a gas channel for a gas connection from the gas-permeable layer to the cavity.

Clause 51. The method of any of claims 48 to 50, comprising providing an electrically conductive layer on the cap substrate.

Clause 52. The method of claim 51, comprising forming an electrical connection between the electrically conductive layer and the substrate.

Clause 53. The method of any of claims 48 to 52, comprising attaching the substrate and the cap substrate by an electrically conductive portion.

Clause The method of any of claims 48 to 53, comprising providing a Faraday cage with the electrolyte, the first electrode, and the second electrode within the Faraday cage.

Clause 55. The method of any of claims 32 to 54, wherein the target gas is oxygen.

Clause 56. The method of any of claims 32 to 55, wherein the electrolyte comprises a polymer comprising: a silicone, an anion, and a cation, at least one of the cation or the anion covalently bonded to the silicone.

Clause 57. The method of any of claims 32 to 56, wherein the cation and the anion when isolated in combination form an ionic liquid.

Clause 58. The method of any of claims 32 to 57, wherein the polymer comprises a linker covalently bonded to the silicone and at least one of the cation or anion.

Clause 59. The method of any of claims 32 to 58, wherein at least one of the anion or the cation comprises at least one of: a carbon-carbon covalent bond, or a carbon-hydrogen covalent bond.

Clause 60. The method of any of claims 32 to 59, wherein the polymer is cross-linked.

Clause 61. The method of any of claims 32 to 60, wherein the first electrode is a working electrode and the second electrode is a counter electrode.

Clause 62. The method of any of claims 32 to 61, wherein the first electrode is a first working electrode and the method comprises providing a second working electrode in contact with a third surface of the electrolyte, the third surface not overlapping with and spaced from the first surface and the second surface.

Clause 63. The method of claim 62, wherein the target gas is a first target gas, the first working electrode is for sensing a first target gas and the second working electrode is for sensing a second target gas different to the first gas.

Clause 64. The method of any of claims 32 to 63, comprising at least one of: providing a reference electrode in contact with the electrolyte; providing a diagnostic electrode in contact with the electrolyte; or providing a grounding electrode in contact with the electrolyte.

Clause 65. The method of any of claims 32 to 64, wherein upon application of the potential difference between the first electrode and the second electrode, an electrical current from the first electrode to the second electrode is no more than 1 Milliampere.

Clause 66. A microfabricated gas sensor obtained by the method of any of claims 32 to 65.

Clause 67. A device comprising the microfabricated gas sensor of any of claim 1 to 30 or 66, and at least one of: a die; a housing at least partly enclosing the gas sensor; a printed circuit board; flexible printed circuit connector; a wafer layer chip scale package; a multiplexer; an operational amplifier; a microcontroller; a power regulator; an application-specific integrated circuit (ASIC); an ASIC, the gas sensor and the ASIC both on a board; an ASIC integrated with the gas sensor; a power management integrated circuit; a communications module; memory; a secondary sensor; or an analogue-to-digital converter.

Clause 68. A method of sensing the concentration of the target gas at the first surface using the microfabricated gas sensor of any of claim 1 to 31 or 66, or the device of claim 67, the method comprising: applying the potential difference between the first electrode and the second electrode; measuring an electrical current value between the first electrode and the second electrode; and determining the concentration of the target gas from the electrical current value.

Clause 69. A method of sensing the partial pressure of the target gas at the first surface using the microfabricated gas sensor of any of claim 1 to 31 or 64, or the device of claim 65, the method comprising: applying the potential difference between the first electrode and the second electrode; measuring an electrical current value between the first electrode and the second electrode; and determining the partial pressure of the target gas at the first surface from the electrical current value.

Clause 71. The method of claim 68 or claim 69, wherein the gas sensor is at least partly submerged or submerged in a liquid.

Clause 72. The method of any of claims 68 to 71, wherein a humidity at the gas sensor is from 0 percent to 100 percent.

Clause 73. The method of any of claims 68 to 72, wherein the temperature of the gas sensor is at least one of: from 194 Kelvin to 573 Kelvin, from 220 Kelvin to 573 Kelvin, or from 220 Kelvin to 453 Kelvin.

Clause 74. Use of the microfabricated gas sensor of any of claim 1 to 31 or 66, or the device of claim 67.

Clause 75. The use of claim 74, wherein the gas sensor at least partly submerged or submerged in a liquid.

Clause 76. The use of claim 74 or claim 75, wherein a humidity at the gas sensor is from 0 percent to 100 percent.

Clause 77. The use of any of claims 74 to 76, wherein the temperature of the gas sensor is at least one of: from 194 Kelvin to 573 Kelvin, from 220 Kelvin to 573 Kelvin, or from 220 Kelvin to 453 Kelvin.

Clause 78. The use of any of claims 74 to 77, comprising sensing a concentration of at least one of: oxygen; carbon dioxide; carbon monoxide; ethanol; hydrogen sulphide; ammonia; NOx; ethylene; chlorine; ozone; hydrogen chloride; hydrogen peroxide; phosphine; hydrogen cyanide; sulphur dioxide; water/humidity; VOCs; hydrogen; or a fuel.

Clause 79. The use of any of claims 74 to 78, comprising sensing a partial pressure of at least one of: oxygen; carbon dioxide; carbon monoxide; ethanol; hydrogen sulphide; ammonia; NOx; ethylene; chlorine; ozone; hydrogen chloride; hydrogen peroxide; phosphine; hydrogen cyanide; sulphur dioxide; water/humidity; VOCs; hydrogen; or a fuel.

Clause 80. The use of any of claims 74 to 79, wherein the use is for at least one of: monitor food; monitor sealed packages or enclosed spaces; breath sensing; monitor breathable atmospheres; environmental monitoring; monitor respiration; fire detection; fitness monitoring; indoor farming; hydrogen storage; monitoring dissolved gases in liquids; hydrogen internal combustion engines; oven pyrolysis; a switchgear; air quality measurement; electrolysis; an electrolyser; or fuel storage.

Clause 81. A method of calibrating a microfabricated gas sensor of any of claim 1 to 31 or 66.

Clause 82. A method of calibrating a plurality of microfabricated gas sensors each of any of claim 1 to 31 or 66.

Clause 83. The method of claim 82, wherein the plurality of microfabricated gas sensors are each supported by a wafer.

Clause 84. The method of claim 83, comprising: (i) with the wafer exposed to at least one of: a first concentration of the target gas, or a first partial pressure of the target gas, for each microfabricated gas sensor of the plurality of microfabricated gas sensors, respectively measuring a first electrical current between the respective first electrode and second electrode; and (ii) with the wafer exposed to at least one of: a second concentration of the target gas, or a second partial pressure of the target gas, for each microfabricated gas sensor of the plurality of microfabricated gas sensors, respectively measuring a second electrical current between the respective first electrode and second electrode.

Clause 85. The method of claim 84, comprising, for each of the microfabricated gas sensors of the plurality of microfabricated gas sensors, determining a respective calibration value, the calibration value for each of the microfabricated gas sensors related to at least one of: the respective first electrical current; or the respective second electrical current.

Clause 86. The method of any of claims 83 to 85, comprising exclusion listing of each microfabricated gas sensor of the plurality of microfabricated gas sensors wherein at least one of: the respective first electrical current is greater than a first threshold; the respective first electrical current is less than a second threshold; the respective second electrical current is greater than a third threshold; or the respective second electrical current is less than a fourth threshold.

Clause 87. The method of any of claims 83 to 86, wherein the plurality of microfabricated gas sensors comprises no fewer than at least one of: 100 microfabricated gas sensors, 1000 microfabricated gas sensors, or 10000 microfabricated gas sensors.

What is claimed is:

1. A microfabricated gas sensor for a target gas, comprising:
an electrolyte at least partly provided using photolithography;
a first electrode, a surface of the first electrode in contact with a first surface of the electrolyte, the surface of the first electrode from 78.5 micrometres squared to 7,850 micrometres squared, and the first electrode for reducing or oxidising the target gas received through the electrolyte to produce target ions; and
a second electrode, a surface of the second electrode in contact with a second surface of the electrolyte, and the surface of the second electrode (106) from 78.5 micrometres squared to 7,850 micrometres squared,
wherein with a potential difference applied between the first and second electrodes an amount of electrical current between the first electrode and the second electrode is indicative of at least one of: a concentration of the target gas at the first surface, or a partial pressure of the target gas at the first surface.

2. The microfabricated gas sensor of claim 1, wherein the electrolyte is inorganic.

3. The microfabricated gas sensor of claim 1, wherein the electrolyte is solid-state.

4. The microfabricated gas sensor of claim 1, wherein the electrolyte is at least partly patterned by photolithography.

5. The microfabricated gas sensor of claim 1, wherein the first surface of the electrolyte and the second surface of the electrolyte do not overlap and are spaced from each other.

6. The microfabricated gas sensor of claim 1, comprises a substrate supporting the electrolyte, the first electrode, and the second electrode; and wherein providing the electrolyte using photolithography comprises: providing a precursor; providing a photomask on the precursor; and irradiating the precursor to form the electrolyte.

7. The microfabricated gas sensor of claim 1, comprising:
a substrate supporting the electrolyte, the first electrode, and the second electrode;
a cap; and
a cavity between the cap and a sensing surface of the electrolyte, the cap comprising: a cap substrate attached to the substrate; and a gas channel from the cavity and through the cap substrate.

8. The microfabricated gas sensor of claim 1, comprising a gas-permeable layer on the cap substrate, the gas channel from the gas-permeable layer to the cavity, wherein the cap comprises an electrically conductive layer between the cap substrate and the cavity, wherein the electrically conductive layer and the substrate are electrically connected, and wherein at least one of:
the substrate and the cap substrate are attached to each other by an electrically conductive portion; or
the cap substrate is electrically conductive.

9. The microfabricated gas sensor of claim 1, comprising a Faraday cage, the electrolyte, the first electrode, and the second electrode within the Faraday cage.

10. The microfabricated gas sensor of claim 1, wherein at least one of:
the electrolyte comprises a polymer comprising: a silicone, an anion, and a cation, at least one of the cation or the anion covalently bonded to the silicone;
the electrolyte comprises a polymer comprising: a silicone, an anion, and a cation, at least one of the cation or the anion covalently bonded to the silicone, and the polymer comprises a linker covalently bonded to the silicone and at least one of the cation or anion; or
the electrolyte comprises a polymer comprising: a silicone, an anion, and a cation, at least one of the cation or the anion covalently bonded to the silicone, and at least one of the anion or the cation comprises at least one of: a carbon-carbon covalent bond, or a carbon-hydrogen covalent bond.

11. The microfabricated gas sensor of claim 10, wherein at least one of:
the polymer is a first polymer, and the electrolyte further comprises a second polymer, and wherein at least one of: the second polymer comprises a silicone; the second polymer comprises a non-silicone; or the second polymer is crosslinked.
the cation comprises at least one of: a triazolium, a thioimidazolium, a quaternary ammonium, a protonated tertiary amine, imidazolium, a substituted imidazolium, a quaternary phosphonium, a protonated tertiary phosphine, chlorinium, pyrrolidinium, a substituted pyrrolidinium, pyridinium, a substituted pyridinium, sulfonium or a substituted sulfonium;
the anion comprises at least one of: a sulfonate, a phosphonate, alkoxide, a thiolate, an imidazolate, tetrakis(pentafluorophenyl) borate, bis(fluorosulfonyl)imide, a tosylate, (methyl)acrylate, a carboxylate, acetate, chloride, bromide, iodide, dimethylphosphate, methylsulfate, hexafluorophosphate, a triflate, bis(trifluoromethane) sulfonimide (bistriflimide/TFSI)), or tetrafluoroborate;
the electrolyte comprises an additive for modification of crosslink density;
the electrolyte comprises a plasticiser;
the electrolyte comprises a redox active additive;
the electrolyte comprises a chromophore;
the electrolyte comprises a surfactant;
the cation is a multi-cation;
the anion is a multi-anion;
the cation is a zwitterion; or
the anion is a zwitterion.

12. The microfabricated gas sensor of claim 1, wherein upon application of the potential difference between the first electrode and the second electrode, an electrical current between the first electrode and the second electrode is no more than 1 Milliampere.

13. The microfabricated gas sensor of claim 1, configured to at least one of:
be used when at least partly submerged or submerged in a liquid to determine at least one of: the concentration of the target gas at the first surface, or the partial pressure of the target gas at the first surface;
be used in a humidity from 0 percent to 100 percent to determine at least one of: the concentration of the target gas at the first surface, or the partial pressure of the target gas at the first surface; or
be used at a temperature at least one of: from 194 Kelvin to 573 Kelvin, from 220 Kelvin to 573 Kelvin, or from 220 Kelvin to 453 Kelvin to determine at least one of: the concentration of the target gas at the first surface, or the partial pressure of the target gas at the first surface.

14. A device comprising a microfabricated gas sensor comprising:
an electrolyte at least partly provided using photolithography;
a first electrode, a surface of the first electrode in contact with a first surface of the electrolyte, the surface of the first electrode from 78.5 micrometres squared to 7,850 micrometres squared, and the first electrode for reducing or oxidising the target gas received through the electrolyte to produce target ions; and
a second electrode, a surface of the second electrode in contact with a second surface of the electrolyte, and the surface of the second electrode (106) from 78.5 micrometres squared to 7,850 micrometres squared,
wherein with a potential difference applied between the first and second electrodes an amount of electrical current between the first electrode and the second electrode is indicative of at least one of a partial pressure of the target gas at the first surface.

15. The device of claim 14, comprising at least one of: a die; a housing at least partly enclosing the gas sensor; a printed circuit board; flexible printed circuit connector; a wafer layer chip scale package; a multiplexer; an operational amplifier; a microcontroller; a power regulator; an application-specific integrated circuit (ASIC); an ASIC, the gas sensor and the ASIC both on a board; an ASIC integrated with the gas sensor; a power management integrated circuit; a communications module; memory; a secondary sensor; or an analogue-to-digital converter.

16. A method of calibrating a plurality of microfabricated gas sensors, each microfabricated gas sensor of the plurality of microfabricated gas sensors respectively comprising:
an electrolyte at least partly provided using photolithography;
a first electrode, a surface of the first electrode in contact with a first surface of the electrolyte, the surface of the first electrode from 78.5 micrometres squared to 7,850 micrometres squared, and the first electrode for reducing or oxidising the target gas received through the electrolyte to produce target ions; and
a second electrode, a surface of the second electrode in contact with a second surface of the electrolyte, and the surface of the second electrode from 78.5 micrometres squared to 7,850 micrometres squared,
wherein with a potential difference applied between the first electrode and the second electrode an amount of electrical current between the first electrode and the second electrode is indicative of at least one of: a concentration of the target gas at the first surface, or a partial pressure of the target gas at the first surface.

17. The method of claim 16, wherein the plurality of microfabricated gas sensors are each supported by a wafer.

18. The method of claim 17, comprising:
(i) with the wafer exposed to at least one of:
a first concentration of the target gas, or
a first partial pressure of the target gas,
for each microfabricated gas sensor of the plurality of microfabricated gas sensors, respectively measuring a first electrical current between the respective first electrode and second electrode; and
(ii) with the wafer exposed to at least one of:
a second concentration of the target gas, or
a second partial pressure of the target gas,
for each microfabricated gas sensor of the plurality of microfabricated gas sensors, respectively measuring a second electrical current between the respective first electrode and second electrode.

19. The method of claim 18, comprising, for each of the microfabricated gas sensors of the plurality of microfabricated gas sensors, determining a respective calibration value, the calibration value for each of the microfabricated gas sensors related to at least one of: the respective first electrical current; or the respective second electrical current.

20. The method of claim 19, comprising exclusion listing of each microfabricated gas sensor of the plurality of microfabricated gas sensors wherein at least one of: the respective first electrical current is greater than a first threshold; the respective first electrical current is less than a second threshold; the respective second electrical current is greater than a third threshold; or the respective second electrical current is less than a fourth threshold.
